(19) United States
(12) Patent Application Publication
(10) Pub. No.: US 2025/0137024 A1
(43) Pub. Date: May 1, 2025

(54) PLASMID DNA PURIFICATION METHODS

(71) Applicant: GLAXOSMITHKLINE BIOLOGICALS SA, Rixensart (BE)

(72) Inventors: Olivier LE BUSSY, Rixensart (BE); Baptiste PIERMAN, Rixensart (BE); Marc ORVAL, Rixensart (BE); Delphine JASON, Rixensart (BE)

(73) Assignee: GLAXOSMITHKLINE BIOLOGICALS SA, Rixensart (BE)

(21) Appl. No.: 18/683,633

(22) PCT Filed: Sep. 1, 2022
(86) PCT No.: PCT/IB2022/058236

§ 371 Date: Feb. 14, 2024

Related U.S. Application Data
(60) Provisional application No. 63/239,545, filed on Sep. 1, 2021.

(51) Int. Cl. C12P 19/34; C07K 14/005; C12N 15/10; C12N 15/85

(52) U.S. Cl. CPC C12P 19/34; C07K 14/005; C12N 15/101; C12N 15/85; C12N 2710/16052; C12N 2760/18552; C12N 2760/20152; C12N 2770/20052; C12N 2800/10

(57) ABSTRACT

This application discloses a method for purifying pDNA, particularly pDNA that that can be used to produce RNA, the RNA preferably encoding a therapeutic or immunogenic peptide or polypeptide. The pDNA can be grown in a bacteria such as E. coli by culturing or fermenting bacteria containing the plasmid and obtaining and purifying the pDNA. The present method allows the pDNA to be obtained in high yield and with high purity. In one embodiment of the invention, the level of all non-pDNA materials can be significantly reduced by the process. In some embodiments, the ratio of supercoiled plasmid DNA (scDNA) to non-supercoiled pDNA (non-scDNA, such as open circular plasmid DNA (ocDNA)) can be increased by one or more process steps that separate or allow for separation of scDNA and ocDNA or process steps that increase the amount of scDNA to ocDNA.

### CROSS-REFERENCE TO RELATED APPLICATION

This application claims priority to U.S. Provisional Application No. 63/239,545 filed on Sep. 1, 2021, then entirety of which is incorporated by reference herein.

### FIELD OF THE INVENTION

The present invention is directed to the field of purification of plasmid DNA (pDNA) and in particular, methods for purification of pDNA which is to eventually be used for research, diagnostic, therapeutic or pharmaceutical purposes or which is to be used for production of RNA that encodes a useful protein, such a as a therapeutic or immunogenic protein suitable for pharmaceutical or research and development use.

### BACKGROUND TO THE INVENTION

Techniques for purification of pDNA have been known for many years. A review of chromatography techniques that can be uses in pDNA purification is provided by Diogo et al, “Chromatography of plasmid DNA”, J. Chromatogr. A, Vol. 1069, pp. 3-22 (2005). Prazeres et al also describe a process for preparative purification of scDNA, Prazeres et al, “Preparative Purification of supercoiled plasmid DNA using anion-exchange chromatography”, J. Chromatogr. A, Vol. 806, pp. 31-45 (1998). However, there is a need for pDNA purification processes that can produce highly pure pDNA (suitable for pharmaceutical uses) that are suitable for production of suitable quantities of pDNA for pharmaceutical uses and for production of pharmaceutical products such as RNA, mRNA or SAM vaccines.

### SUMMARY OF THE INVENTION

The present invention is directed to a method for purifying pDNA, particularly pDNA that encodes a therapeutic or immunogenic peptide or polypeptide and more particularly a pDNA that that can be used to produce an RNA molecule, more specifically an mRNA molecule or a self-amplifying mRNA (SAM) molecule, the RNA, mRNA or SAM molecules preferably encoding a therapeutic or immunogenic peptide or polypeptide. The pDNA can be produced in a bacteria such as E. coli by culturing or fermenting the microorganism containing the plasmid and purifying the pDNA. The present method allows the pDNA to be obtained in high yield and with high purity.

In some embodiments of the invention, the plasmid DNA purification method is optimized to produce high quantity and quality yields of purified pDNA as compared to other commercial kit processes defined in the art. In one embodiments of the invention, the level of all non-pDNA materials can be significantly reduced by the process of the present invention.

In another embodiment, the ratio of supercoiled plasmid DNA (scDNA) to non-scDNA can be increased by one or more process steps that separate or allow for separation of scDNA to non-scDNA (such as open circular plasmid DNA (ocDNA)). In some embodiments, pDNA quality was increased by reduction of impurities due to elimination of key contaminants prior to entry into downstream processing. In some embodiments, pDNA quality was increased by the advantageous selection of the scDNA fraction in AEX.

In another embodiment, the present invention relates to highly purified pDNA and uses thereof.

### DESCRIPTION OF THE DRAWINGS

FIG. 1 is a plasmid production and purification flowchart that shows some of the main aspects of the method of the invention, beginning with lysis of cells harboring the plasmid and resulting, at the end of the process, in highly purified pDNA.

FIG. 2 shows the elution gradient results for the Fractogel EMD DMAE (Run 18-b in Table 5).

FIG. 3 is gel of the four materials reported in the Table 7, wherein lane 1 is a molecular weight control, lane 2 is the load to the DMAE, lane 3 is Elution 1 (corresponding to the peak #1 in FIG. 2, which is almost exclusively ocDNA) and lane 4 is Elution 2 (corresponding to peak #2 in FIG. 2 that is mainly scDNA).

FIG. 4 is a DMAE chromatogram showing the two peaks at elution in a sample containing pDNA encoding HV protein. Peak 1 is mainly ocDNA and Peak 2 is mainly scDNA.

FIG. 5 is a flow chart that summarizes the treatment of scDNA obtained from AEX purification of HVpDNA. Where present, the circled numbers in the upper left corner of the flow chart boxes refer to the correspondingly labelled inset in FIG. 6A-C.

FIGS. 6A-6D report further experiments that characterize the scDNA at various stages of the further treatment steps shown in FIG. 5. FIG. 6A depicts an agarose gel run on eluted pDNA collected from an anion exchange chromatography as described of FIG. 4. “PIC1” refers to eluate emerging in a peak that corresponds to Peak 1, “PIC2” refers to eluate emerging in a peak that corresponds to Peak 2. FIG. 6B depicts the results of digesting the eluted pDNA with S1 nuclease. S1 nuclease cuts double-stranded DNA at single stranded nicks. Thus, plasmid DNA having nicks will be cut at each nick, thus producing a smear when run on agarose gel. PIC1 material produces greater smearing than PIC2, indicating that it has more nicks than PIC2. FIG. 6C depicts an agarose gel run on RNA produced from an in vitro transcription reaction carried out using either the pDNA of PIC1 or the pDNA of PIC2.

FIG. 7 is a graph that shows the ability of various resins to remove endotoxin.

FIG. 8 includes gels that show that the Hepatitis antigen, COVID, Rabies and HSVth plasmid samples that were subjected to further testing moved differently in an agarose gel.

FIG. 9A and FIG. 9B (Hepatitis antigen), FIG. 10A and FIG. 10B (COVID); FIG. 11A and FIG. 11B (Rabies); and FIG. 12A and FIG. 12B (HSVth) show results of further purification of the samples of FIG. 8, and also show results for MW control and a commercial pDNA purification kit.

FIG. 13 is a modification of the flowchart shown in FIG. 1 which illustrates the main aspects of the method described in Example 11 which is directed to modifications to accommodate large batch purification of plasmid DNA.

FIG. 14A and FIG. 14B are graphs displaying data from CaCl₂) screening for the precipitation of RNA from lysate.

FIG. 15 is a DMAE chromatogram showing the two peaks at elution in a large batch sample containing pDNA.

FIG. 16 is a gel showing the plasmid samples on an agarose gel; wherein lane 1 is the load to the DMAE, lane 2 is BreakThrough (BT), lane 3 is Wash 2 (W2), lane 4 is the elution peak 1 (P1) (corresponding to the peak #1 which is almost exclusively ocDNA) and lane 5 is the elution peak 2 (P2) (corresponding to the peak #2 in FIG. 15 that is mainly scDNA).

FIG. 17 is a gel showing the comparison of sc pDNA samples on an agarose gel present at various stages of both the small-scale and large-scale purification method disclosed herein.

### DETAILED DESCRIPTION OF THE INVENTION

Further details of the invention are provided below.

### Definitions and Abbreviations

AEX—Anion Exchange Chromatography
BT—BreakThrough
CV—column volume
ddPCR—Droplet Digital PCR
DMAE—Dimethylethanolamine
DSP—Downstream Process
DV—diafiltration volume
DF—diafiltration
FT—Flow Through
HV—Hepatitis virus
HCP—Host Cell Proteins
HIC— hydrophobic interaction chromatography
HSVth—Herpes simplex Virus therapeutic protein
IVT—In vitro transcription
Kbp—kilo base pair
LS—large-scale. In this application, the term “large-scale” or “larger-scale” refers to the mass of a cell paste used to prepare a cell lysate obtained by lysis of cells containing pDNA in a batch fermentation or cultivation step. A “large-scale” or “larger-scale” batch treat a paste having a mass of greater than 100 grams or greater than 200 grams or greater than 500 grams, typically 200 grams up to 2 kilograms, and even more typically 500 grams up to 2 kg and even more typically 600 grams to up to 1.5 kg. In the Examples below, the large-scale process is performed on a paste having a mass of 750 g.
LMW RNA—low molecular weight RNA, usually less than 200 bp
Mr—The molecular mass (abbreviated Mr) of a substance, formerly also called molecular weight and abbreviated as MW, is the mass of one molecule of that substance, relative to the unified atomic mass unit u (equal to 1/12 the mass of one atom of carbon-12). Due to this relativity, the molecular mass of a substance is commonly referred to as the relative molecular mass, and abbreviated to Mr.
mS/cm—milliSiemens/cm
MWCO—molecular weight cut off
ocDNA—open circular DNA
pDNA—plasmid DNA
P1—peak 1
P2—peak 2
SAM—self-amplifying mRNA
SS—Small-Scale—In this application, the term “small-scale” or “smaller-scale” refers to the mass of a cell paste used to prepare a cell lysate obtained by lysis of cells containing pDNA in a batch fermentation or cultivation step. A “small-scale” or “smaller-scale” batch would treat a paste having a mass of 100 grams or less or less, typically 10 to 100 grams and even more typically 20 to 100 grams. In the Examples below, the small-scale process is performed on a paste having a mass of 50 g.
scDNA—supercoiled DNA
SN—supernatant
TFF—Tangential flow filtration
UF—ultrafiltration
W2—Wash 2
Xto—chromatography
   The pDNA

pDNA has many uses including research, molecular biology, diagnostic, manufacturing and therapeutic or pharmaceutical uses. pDNA is also useful for production of RNA that encodes a useful protein, such a as a therapeutic or immunogenic protein suitable for pharmaceutical use. One use of pDNA at the present time is for the production of RNA, such as mRNA such as self-amplifying mRNA (SAM). In some embodiments, pDNA finds use in the making of a conventional mRNA, which is often transcribed from DNA and plasmids. In this regard, it is understood that the convention mRNA may include one or more of a cap, a 5′ untranslated region (UTR), a sequence that encodes a therapeutic or immunogenic protein, a poly-A region, and a 3′ UTR. In some embodiments, the pDNA finds use in the making of SAM. In this regard, it is understood that a SAM can comprise one or more of a cap, a 5′ untranslated region (UTR), a sequence that encodes a therapeutic or immunogenic protein, a poly-A region, and a 3′ UTR, as well as one or more sequences that encode one or more proteins necessary for replicating the SAM in an intracellular environment, and that these segments or sequences that encode the one or more proteins necessary for replicating the self-replicating RNA. In some embodiments, the mRNA, such as conventional mRNA and/or SAM, finds use for the production of an mRNA vaccine that encodes an immunogen that can elicit an immunogenic response against a pathogen such as a virus.

In a broad sense, the pDNA that can be purified in accordance with the present invention is any pDNA, including pDNA of various sizes. pDNA that can be purified in accordance with the present invention will usually have a size between 3 and 20 kbp (kilo base pairs). When the pDNA encodes “conventional” RNA, the pDNA will usually have a size in the lower end of this range, such as 3-8 kbps, such as 3-7, 3-6 or 3-5 kbps. When the pDNA encodes SAM, the pDNA will usually have a size in the upper end of this range, such as 8-20 kbps, such as 9-18, or 10-16 or 12-14 kbps. It is noted that the plasmid sizes for “conventional” mRNA tested in the below Examples are about 4165 bp and the “empty” plasmid size is 2265. For plasmids encoding OMICRON mRNA, the plasmid size is 6250 bp and the empty plasmid size is also 2265.

Depending upon plasmid quality and the conditions in which it exists (the properties of the buffer or solution in which is dissolved or suspended), the pDNA, particularly pDNA produced in E. coli, can take various three-dimensional shapes and exhibit various physical properties. For example, pDNA can exist in a negatively charged supercoiled (sc) form that exhibits a long, thin and branched structure or can exist in a relaxed or open circular (oc) form, with no coiling of the double helix. Stated another way, the supercoiled form is much more twisted upon itself than the open circular form which is more relaxed. So the negative charge, bared by the phosphodiester groups of the DNA backbone, is more dense in the sc form while in the oc form the negative charge is more dispersed. This results in a difference in local charge density between the two forms that modulate a difference in the electrostatic attractions to positively charged ligands of resins used in plasmid purification or separation steps. More information concerning pDNA purification and separation can be found in Prazeres et al, “Preparative Purification of supercoiled plasmid DNA using anion-exchange chromatography”, J. Chromatogr. A, Vol. 806, pp. 31-45 (1998).

When dealing with pDNA that encodes a SAM molecule, pDNA molecules that are in the supercoiled form because this form is usually associated with high quality pDNA, whereas ocDNA is an indicator that the pDNA has some sort of a defect such as a nick, which is undesirable.

The pDNA to be purified may have a size of 3 to 20 kbp. When the pDNA encodes SAM which encodes a viral immunogen, the pDNA size is likely to be at the higher end of this range such as 10 to 20 kbp or 12 to 18 kbp, and likely 13 to 17 or 14 to 16 kbp. A typical size pDNA encoding SAM that encodes a viral antigen is 14.5 to 15.5 kbp or 15 kbp. With larger pDNA, such as pDNA encoding SAM which encodes a viral immunogen, it may be more likely for the plasmid to get a “nick” in it, thus creating a certain amount of ocDNA in addition to the desired scDNA.

The final product of the process of the present invention (after the AEX step and/or also at the end of the described process) is ideally at least 80% pure. Therefore, in purified scDNA produced in the present invention will ideally have a ratio ocDNA/scDNA less than or equal to ¼ (greater than or equal to 80% scDNA), preferably less than ⅕, more preferably less than 1/10 and most preferably less than 1/20.

The pDNA may encode a SAM molecule that encodes any desired an antigen or protein for a therapeutic or immunogenic use. The antigen or protein can be of viral or bacterial origin (including modifications, derivatives and/or fragments of natural antigens or proteins) or can be antigens or proteins useful for treating an infectious disease. Examples of antigens or proteins that are encoded by SAM molecules used in the Examples are as follows:
HV antigen—Hepatitis virus antigen
COVID—a spike protein from a COVID virus such as COVID-19.
Rabies—an immunogenic protein or peptide from rabies virus or for treating rabies virus infection. See, for example, WO 2019/016680 A1.
HSVth—a therapeutic protein for treating Herpes Simplex virus infection. See, for example, WO 2021/013798 A1.

### Overall Process Conditions

The process of the present invention is described in the Examples in terms of a batch process. The majority this batch process (for all filtration and centrifugations steps) is conducted under open but sterile conditions in order to ensure quality control. A batch process will usually be sufficient because a bulk amount of high-quality plasmid DNA can be prepared and then stored for future use. However, the process can be adapted to a continuous process.

Individual process steps can be optimized based on the size, charge and other properties of the particular pDNA being purified, taking into account the teachings in the present application.

### Lysis

In this step, the cells containing the plasmid DNA to be purified are lysed. Typically, a cell paste is obtained and this cell paste is then subjected to lysis. Alkaline lysis is particularly suitable for lysing E. coli cells. Various alkaline lysis procedures are known in the art as reported, for example, by D. M. F. Prazeres, et al, Journal of Chromatography A, 806 (1998) 31-45, “Preparative purification of supercoiled plasmid DNA using anion-exchange chromatography” and by M. M. Diogo, et al, Journal of Chromatography A, 1069 (2005) 3-22, “Chromatography of plasmid DNA”.

In the procedures reported in the Examples of this application, the host cells are bacterial cells, typically gram-negative bacterial cells such as E. coli cells. If cells other than gram negative cells such as E. coli cells are used to produce (or multiply or increase the quantity of) the plasmids, the early steps in the process such as lysis conditions will likely be modified in order to optimize the process and other steps might have different functions, or might not be necessary or might have different purposes because of the absence of endotoxins.

### Neutralization

One purpose of this step is to neutralize the alkali added in the previous alkaline lysis step. This step also precipitates gDNA, HCPs and cell debris. Many of the HCPs will co-precipitate together with the cell debris due to complexation with anionic surfactant (SDS used in the Examples) added in the lysis solution. Various neutralization procedures are known in the art as reported, for example, by D. M. F. Prazeres, et al, Journal of Chromatography A, 806 (1998) 31-45, “Preparative purification of supercoiled plasmid DNA using anion-exchange chromatography” and by M. M. Diogo, et al, Journal of Chromatography A, 1069 (2005) 3-22, “Chromatography of plasmid DNA”.

### Clarification

The purpose of the clarification step is to further purify the materials that are still in the pDNA containing sample. This step may involve, for example, precipitation of HMW RNA and genomic DNA (gDNA). In this step, neutralized cell lysate obtained from the neutralization step is clarified by precipitation, centrifugation and filtration to prepare a clarified composition. Various clarification steps are known in the art as reported, for example, by D. M. F. Prazeres, et al, Journal of Chromatography A, 806 (1998) 31-45, “Preparative purification of supercoiled plasmid DNA using anion-exchange chromatography” and by M. M. Diogo, et al, Journal of Chromatography A, 1069 (2005) 3-22, “Chromatography of plasmid DNA”.

In this clarification step, CaCl₂) is added directly to the neutralized cell lysate of to bring the CaCl₂) concentration to a desired concentration. In the Examples reported below, a CaCl₂) concentration between 0.2 and 0.6 M, preferably 0.3 and 0.5 M and preferably about 0.4 M was considered optimal in terms of obtaining a max Q DNA and min ratio of RNA/DNA. A single centrifugation step can be employed in this clarification step. In some processes, two centrifugation steps are needed at this stage and the single centrifugation step that can be employed in accordance with the present invention simplifies the process.

### Tangential Flow Filtration

According to the invention, tangential flow filtration (TFF) may be used to purify a pDNA of interest by removing lower molecular weight species and also to reduce the volume of the sample for the forthcoming (downstream) next process step.

A method of the invention can comprise one or more steps of TFF. TFF is particularly useful for the purification of large pDNA species. The inventors have shown that yields of pDNA for the 2^{nd} UF (which is part of this step) can be as high as 90% or greater, such as 90-95%. No measure available for the 1^{st} UF as no plasmid specific assay was available to the inventors. High yields of pDNA such as 90% or greater or 90-99.9%) are therefore considered to be part of the invention for the TFF step, while retaining the stability and potency of the purified pDNA. Usefully, TFF also permits buffer exchange (dialysis) at the same time as purification (or TFF can be used with purified pDNA as a separate buffer exchange step e.g. to change to a final formulation buffer. TFF is easy to operate, time-efficient (only about 70 minutes for both pDNA purification and buffer exchange) and prevents contamination due to the ability to operate as a closed system.

TFF involves passing a liquid containing the sample tangentially across a filter membrane. Thus, TFF contrasts with dead-end filtration, in which sample is passed through a membrane rather than tangentially to it. In TFF the sample side is typically held at a positive pressure relative to the filtrate side. As the liquid flows over the filter, components therein can pass through the membrane into the filtrate. In this step, typical components to be remove include host cell proteins and small RNA fragments, ions in solution, and/or other undesired components. These components are typically removed in the filtrate whereas long pDNA is recovered from the retentate. Many TFF systems are commercially available (e.g. using hollow fibres such as those available from GE Healthcare and Spectrum Labs or flat-sheet cassettes from Merck-Millipore). The molecular weight cut-off (MWCO) of a TFF membrane determines which solutes can pass through the membrane (i.e. into the filtrate) and which are retained (i.e. in the retentate). The MWCO of a TFF filter used with the invention will be selected such that substantially all of the solutes of interest (i.e. desired pDNA species) remains in the retentate, whereas undesired components pass into the filtrate. The retentate may be re-circulated to the feed reservoir to be re-filtered in additional cycles. Compared to dead-end filtration, the retentate is washed away during the filtration process, minimising the clogging of the membrane which is known in the art as “membrane fouling”, maintaining a high, steady filtration rate across the membrane, and increasing the length of time the process can be continuously operated.

The use of TFF in RNA purification methods is disclosed in WO 2014/140211. These procedures can be adapted for purification of pDNA in accordance with the present invention. Parameters for operating TFF according to this invention will be selected such that impurities can permeate the filter membrane whereas the pDNA of interest is retained, without significantly affecting pDNA integrity and/or potency.

The average pore size of a filter membrane is referred to in the art as “membrane pore size”. Membrane pore size is usually stated in kDa and refers to the average molecular mass of the smallest particle or macromolecule the membrane is likely to retain. Alternatively, membrane pore size can be stated in μm and refers to the diameter of the smallest particle the membrane is likely to retain. The diameter is proportional to the molecular mass for molecules of a similar shape (e.g. spherical molecules). For example, a membrane pore size of 500 kDa is equivalent to a membrane pore size of approximately 0.02 μm for a spherical molecule.

The inventors have found that a membrane with a cut off of 30 kDa to 50 kDa is suitable. If a cut off of above 100 kDa is used, significant amounts of pDNA is lost in the permeate. Thus, in accordance of the present invention, a membrane with a cut off pore size of 100 kDa or less is typically used, preferably less than 100 kDa, more preferably 75 kDa or less, even more preferably 10 kDa but less than 100 kDA, 10 to 60 kDa or 20 to 60 kDa or 30 to 60 kDa, or between 10 and 50 kDa, 20 to 5o kDa or 30 and 50 kDa. Ideal cut off sized may be different for different size plasmids. It should be noted that pDNA are linear and very flexible (elastic) and their flexibility is relative to the ionic strength of the buffer. The ideal cut off value for this step can be set experimentally for given solutions to be treated within the above guidelines.

Where a sample comprises a desired pDNA and a non-desired pDNA species of a different size, the method may include two or more steps of tangential flow filtration, wherein each step uses a different membrane pore size such that in one step smaller molecules than the pDNA of interest are removed and the pDNA-containing retentate fraction is retained, and in one or more additional steps larger molecules than the pDNA of interest are removed and the pDNA of interest is recovered from the filtrate.

TFF may be carried out using any suitable filter membrane. The inventors have found that a flat sheet cassette is particularly advantageous. Hollow fibre filters may also be used if desired. A hollow fibre filter typically comprises a multitude (bundle) of hollow, open-ended tubes (fibres), through which the liquid containing the sample is passed from the feed side to the retentate side. The walls of the tubes are composed of a membrane (the filter membrane), which typically has a three-dimensional internal structure of interconnected cavities (pores).

Various common filter membrane polymers (for flat sheet cassette membranes or hollow fibre filters) can be used in this invention. Possible membranes, in cassettes, include regenerated cellulose (RC) cassettes and PES cassettes. While RC is more hydrophilic than PES, PES is more chemically resistant as built material, so the flux performance and cleanability of the PES membrane were better compared to RC membrane. Thus, suitable polymers (some of which are more typically used in hollow fibers) may include regenerated cellulose, polysulfone (PS), polyethersulfone (PES). PES may be modified (mPES) to have increased hydrophilicity and to have higher permeate flux rates than un-modified PES. Several different methods are known to transform hydrophobic PES membranes into hydrophilic PES membranes.

A TFF method may vary according to the transmembrane pressure that is applied during the process. Transmembrane pressure is the average pressure differential between the feed side and the filtrate side of the filter membrane. Ideally, the transmembrane pressure is chosen so that a high flux of the fluid across the membrane is achieved while maintaining efficient separation of the pDNA of interest from any impurities and avoiding the formation of a gel layer on the surface of the filter membrane. The inventors have found that a transmembrane pressure between 4 psi (27579 Pa) and 6 psi (41368 Pa) is preferred. Ideally, the transmembrane pressure is set to about 5 psi (34474 Pa).

A fluid may be fed into the TFF system in addition to the pDNA-containing sample. The fluid is typically a buffer. The choice and composition of the buffer may influence the efficiency of pDNA purification and/or buffer exchange, levels of protein aggregation, pDNA-protein separation and pDNA stability. Typical buffer include those based on Tris. The inventors have found that a Tris based buffer, for example containing 10 mM Tris, performs particularly well. Preferably, the buffer pH is between 6.0 and 9.0, between 6.5 and 9.0, between 7.0 and 8.5, between 7.0 and 8.0, between 7.3 and 7.8. More preferably, the sample buffer pH is 7.5.

However, the inventors have found that excessive salt concentration in the buffer should ideally be avoided due to the risk of pDNA precipitation during TFF or disadvantageous effects in any downstream methods. It is therefore preferred that no salt, other than buffering salts, is added to the buffer for TFF purification. Addition of EDTA to a buffer is known to advantageously inhibit any RNase activity.

The volume ratio of the additional fluid (i.e. the fluid which is added beyond that of the sample) may influence the efficiency of the removal of small molecules during pDNA purification and/or buffer exchange. However, larger volumes increase the operation time. Typically, the volume ratio of the additional fluid to that of the sample is between 5:1 and 30:1. The inventors have found that a ratio of above 10:1 or above 15:1 is useful to improve clearance of LMW RNA. In particular, a ratio of above 20:1 such as 20:1 to 30:1 is preferred to ensure efficient purification and/or buffer exchange without unduly increasing the operation time.

### Core Bead Flow-Through Chromatography

The purpose of this step is to remove LMW RNA and reduce HCP and endotoxins. This is achieved by using core bead flow-through chromatography which allows the pDNA to be recovered to pass through the core bead flow-through chromatography and to have as much of low MW impurities such as LMW RNA, HCP and endotoxins to pass through the pores of the chromatography material and be retained inside the chromatography material (such as inside beads).

According to the invention, pDNA may be purified using core bead flow-through chromatography (sometimes also referred to as multimodal exchange chromatography). Thus, a method of the invention can comprise one or more steps of core bead flow-through chromatography. The inventors have found that this technique enables a fast, industrial-scale purification process for obtaining pure pDNA with high yield, and is particularly advantageous for removing protein contaminants from a desired pDNA species e.g. in a reaction sample derived from lysis of cells containing pDNA. The inventors have shown that very large pDNA species comprising more than 3 megadaltons may be purified using this method. However, this method is not limited to the purification of large pDNA molecules, and pDNA molecules of any size (e.g. medium pDNAs) can be purified with this method as long as a suitable bead pore size is selected, as described below.

Core bead flow-through chromatography may be performed using a batch format or a column format. A column format is preferred. The column comprises the stationary phase. The column format may include applying a pDNA-containing sample to the column, collecting the flow-through, and optionally passing elution buffer through the column, and collecting the desired eluates or fractions thereof. The method may comprise additional steps such as wash steps e.g. after applying the sample to the column, a “chase” buffer is usually added to the column. Suitable chromatography setups are known in the art, for example liquid chromatography systems such as the ÄKTA liquid chromatography systems from GE Healthcare (more recently called Cytiva).

After applying the pDNA-containing sample to the column, its contents can travel through the column by gravitational force alone or external pressure may be applied to increase the rate of their passage. Following application of the pDNA-containing sample to the column, a buffer may also be applied to the column, typically called a “chase buffer” in the art, and passed through the column using gravitational force alone or by applying external pressure in order to increase the rate at which the sample components pass through the column. The flow rate can be stated as volumetric flow rate (volume of mobile phase, e.g. sample and/or chase buffer, passing through the column per unit time) or linear flow rate (distance of mobile phase front travelled per unit time). Methods to calculate the flow rate and convert from linear to volumetric flow rate are known in the art.

According to the invention, the chromatography medium is comprised of beads that are comprised of a porous material (matrix), usually formed from a polymer. The matrix comprises at least two layers, for example an inner layer (core) surrounded by an outer layer (shell), but the matrix may also comprise one or more additional (intermediate) layers between the inner layer and the outer layer.

Each matrix layer may be functionalised with at least one ligand, or it may not be functionalised. Typically, the layers can be distinguished from each other by the presence or absence of at least one ligand.

For example, the core may be functionalised with N different ligands, whereas the shell is functionalised with no more than N-1 of these ligands. N may be any positive integer, for example 1. For example, the core may be functionalised with a ligand whereas the shell is functionalised with one or more different ligands, or may not be functionalised with any ligand. In a preferred embodiment, the core is functionalised with a ligand, whereas the shell is not functionalised with any ligands.

Preferably, at least one ligand is a ligand that has multiple functionalities, for example the ligand is both hydrophobic and positively charged. For example, the ligand may be a mono-(C¹-C⁸)alkyl-amine, for example the ligand may be octylamine (CH₃(CH₂)₇NH₂).

Thus in a preferred embodiment of the invention, the core is functionalised with a ligand, wherein the ligand has multiple functionalities, for example the ligand is both hydrophobic and positively charged, for example the ligand may be a mono-(C₁-C₈)alkyl-amine, for example the ligand may be octylamine, and the shell is not functionalised with any ligands.

The matrix has a defined pore size and thereby prevents a proportion of molecules from entering the core based on the size of the molecules, which are collected in the column flow-through (flow-through mode). Molecules that are able to pass through the matrix enter the core, where they may be retained, typically by binding to a ligand. Retained molecules may be eluted from the beads using a suitable eluent (bind-elute mode). Typically, the eluent is a solution comprising sodium hydroxide (NaOH) and a solvent.

Core bead flow-through chromatography parameters will be selected such that a desired pDNA can be selectively recovered from one or more of the flow-through fraction(s), without significantly affecting pDNA integrity and/or potency.

Preferably, the matrix is a porous matrix, for example agarose, preferably a highly cross-linked agarose.

The matrix pore size is usually stated in kDa and refers to the average molecular mass of the smallest particle the matrix is likely to reject (also referred to as MWCO). Alternatively, the matrix pore size can be stated in μm and refers to the diameter of the smallest particle the matrix is likely to reject, as described above for TFF. The pore size is selected so that the cut-off is below pDNA size but above protein size. Using this method, pDNA species may be purified that are larger than the molecular cut-off of the beads. More preferably, the desired pDNA species is the largest molecule in the sample to be purified. Therefore, according to this invention pDNA is recovered from one or more of the flow-through fraction(s).

The inventors have found that for the purification of large pDNAs, a MWCO/pore size of at least 250 kDa is useful e.g. at least 300 kDa, 400 kDa, 500 kDa, 600 kDa, or at least 700 kDa etc. A molecular weight cut-off of at least about 700 kDa is particularly preferred for the pDNA samples used in the Examples that follow. In these Examples, considering the size of the pDNA to be recovered, MWCOs of 300 kDa or 400 kDa and a MWCO of 700 kDa is suitable.

The average diameter (particle size) of the beads will be selected so to enable efficient pDNA purification with minimal operation time without significantly affecting pDNA integrity and/or potency due to excessive pressures required for performance. Larger particles and larger pores typically allow the use of lower pressures, but the separation efficiency may be reduced. The inventors have found that a particle size of about 50-100 μm is preferable, wherein a particle size of about 60-90 μm is more preferably, and wherein a particle size of about 70-80 μm is even preferable. A particle size of about 85 μm is most preferred.

An exemplary core bead flow-through chromatography medium is Capto™ Core 700 beads from GE Healthcare.

pDNA is selectively recovered from the column in the flow-through. Proteins and short nucleic acids are retained in the beads. Flow-through fractions containing pDNA may be identified by measuring UV absorption at 260 nm. The composition comprising the pDNA of interest collected in the flow-through is highly purified relative to the preparation before the core bead chromatography step. Multiple eluted fractions containing the pDNA of interest may be combined before further treatment.

An amount of a salt may be added to the pDNA-containing sample before the sample is passed through the column. The inventors have found that this is particularly advantageous for the removal of protein and endotoxin impurities. Any suitable salt may at a suitable concentration be used, for example at between about 150 mM and 900 mM.

For example, a salt may be added to the pDNA-containing sample to produce a final concentration of between 150 mM to 900 mM, preferably greater than 250 to less than 750 mM, more preferably 300 mM to 700 mM and even more preferably 400 mM to 600 mM. The inventors have found that a salt concentration of about 500 mM (0.5 M) of NaCl is optimal for the recovery of pDNA encoding Hepatitis antigen protein used in the Examples. Selection of an ideal salt concentration is important to achieve pDNA purification with a high pDNA yield and efficient protein removal. Alternatively, where a high pDNA yield is required more than removal of protein impurities, for example where a sample that is substantially free from protein is used, a lower salt concentration such as 0.25 M NaCl can be used.

A suitable salt is typically a salt which minimises the levels of pDNA precipitation compared to less preferred salts when used at the same concentration and pH. The inventors have found that typically sodium chloride is preferred over potassium phosphate, potassium chloride and/or sodium phosphate because sodium chloride salt advantageously minimizes the levels of pDNA precipitation compared to the other identified salts when used at the same concentration and pH.

The flow rate may be varied to achieve improved pDNA recovery and/or protein removal. A linear flow rate of between 200 and 500 cm/h is advantageous where a high pDNA recovery is desired. A flow rate of between 50 and 200 cm/h is advantageous where a high level of protein removal is desired. Typically, a flow rate of between 70 and 150 cm/h, preferably of about 100 cm/h is used for optimised recovery and protein removal.

The addition of a salt, dilution of the sample, and variation of the flow rate, as described above, can usefully be combined. For example, the pDNA-containing sample may be diluted and an amount of a salt may be added to the sample before the sample is passed through the column. A particularly advantageous method for the purification of large pDNA with high purity, yield and short operation times is one where sample is diluted 4-fold before applying the sample to the column, the chromatography is performed at a linear flow rate of 90 cm/h and salt is added to the sample and/or chase buffer at 500 mM (e.g. KCl or NaCl).

Where core bead chromatography is used according to the invention, it is particularly useful for removing protein contaminants from a pDNA of interest. Particularly good results are achieved where the pDNA-containing sample that is applied to the chromatography column in a single purification run contains no more than 5-15 mg total protein per ml of stationary phase (i.e. core beads), e.g. no more than 10 mg/ml or no more than 13 mg/ml. In the Examples, values of from 5 to 7 mg total protein/ml of resin. at Capto Loading was successfully performed. These values are particularly relevant where the total protein is composed of proteins that are typically components of a lysed E. coli sample containing pDNA, such as HCPs, endotoxins and LMW RNA that are present after alkaline lysis of E. coli in preceding steps.) Where large-scale purification is performed, chromatography columns may be connected to each other in series for increased capacity.

### Anion Exchange (AEX)

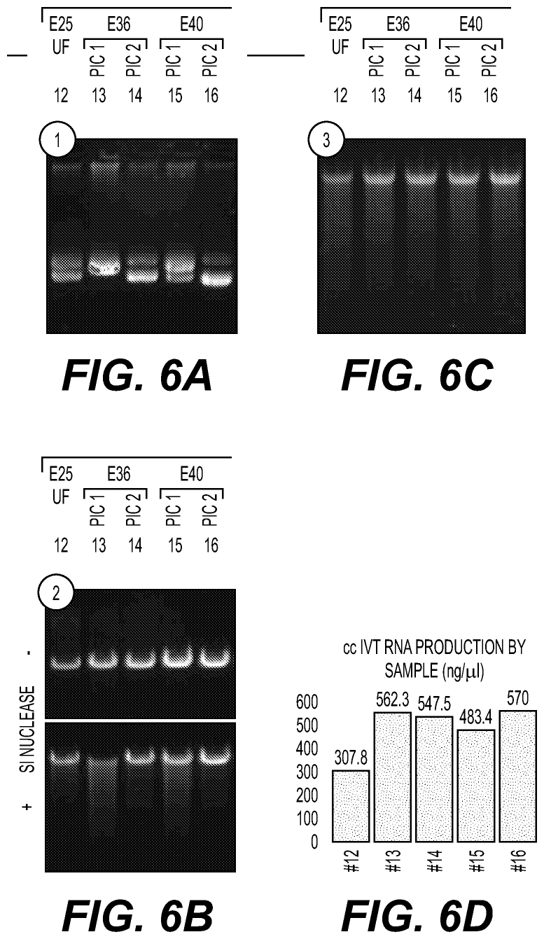

The purpose of this step is to remove (or reduce the amount of) proteins, RNA and endotoxins. In addition, this step separates the following two isoforms of plasmids: (1) supercoiled plasmids (which are twisted and/or coiled on themselves) and (2) open circular plasmids. The supercoiled plasmids are the desired form of the plasmids because they are intact with no breaks in one of the strands. The open circular plasmids have a break or nick in one of the two strands. The inventors have shown in FIG. 6 and FIG. 6B in the drawings that supercoiled plasmid DNA is the desirable isoform. Open circular (or “nicked”) DNA has poor in vitro transcription as compared to supercoiled plasmids and this is why it is desirable to recover as much supercoiled plasmid as possible and remove open circular plasmid.

Various anion exchange materials and methods can be used in this step. Various anion exchange materials are known in the art and include, for example, PRAESTO® Q65, Capto Q, Nuvia Q, GigaCap Q-650M, Fractogel EMD DEAE, Fractogel TMAE Hicap, Fractogel EMD DMAE, Q Sepharose XL, Capto DEAE. Of these, Fractogel EMD DMAE is particularly suitable in terms of yield and contaminant clearance. Fractogel family resins offer better binding capacity for large biomolecules (like pDNA) due to use of a resin with a chemical extender (tentacular resin). Weak anion exchange resins (lke DEAE or DMAE) offer better selectivity compared to strong anion exchanger (like Q ligand) and hence enables sc/oc plasmid separation in conditions used in the experiments reported in this application.

The use of anion exchange materials such as Q Sepharose, DEAE Sepharose FF and Fractogel DEAE in plasmid purification is described in the following references. Use of Q Sepharose to separate OC and SC DNA is described by D. M. F. Prazeres, et al, Journal of Chromatography A, 806 (1998) 31-45, “Preparative purification of supercoiled plasmid DNA using anion-exchange chromatography” and by M. M. Diogo, et al, Journal of Chromatography A, 1069 (2005) 3-22, “Chromatography of plasmid DNA”. Use of DEAE Sepharose FF is described by—H. Li, et al, Cytotechnology (2011) 63: 7-12, “Separation of supercoiled from open circular forms of plasmid DNA, and biological activity detection”. Use of Fractogel DEAE to isolate scDNA (supercoiled DNA) is described by A. Eon-Duval and G. Burke, Journal of Chromatography B, 804 (2004) 327-335, “Purification of pharmaceutical-grade plasmid DNA by anion-exchange chromatography in an RNase-free process”.

A suitable buffer base and conditions to be used in this step with AEX resins is common buffer base: 25 mM Tris—1 mM EDTA—pH 7.5
Spike sample to 0.5 M NaCl
Equilibration & Load at 0.5 M NaCl
Bind pDNA (Protein and endotox pass in FT)
Wash at 0.65M NaCl
Elution with a gradient 0.65M-1.0M NaCl) on 15 CV

The pDNA after the AEX step is ideally at least 80% pure. Therefore, in purified scDNA at the end of this step will ideally have a ratio ocDNA/scDNA less than or equal to ¼ (greater than or equal to 80% scDNA), preferably less than ⅕, more preferably less than 1/10 and most preferably less than 1/20. This level of scDNA purity will continue throughout the rest of the process.

### Hydrophobic Interaction Chromatography (IC)

The purpose of this step is to polish HCPs and endotoxins. This can be accomplished by contacting the material obtained from the AEX step with a HIC resin such as Captobutyl or Sartobind phenyl. For purifying pDNA plasmids in accordance with the present invention, particularly pDNA plasmids of the preferred sizes and other characteristics relevant to the present invention, Captobutyl is preferred over Sartobind phenyl in terms of scalability, yield and flexibility (available surface sizes). Octyl sepharose 4FF, Butyl sepharose 4FF, Phenyl sepharose 6FF, Toyo Butyl-600M, Toyo Butyl-650M, Toyo Phenyl-650M, Capto Butyl and Capto Octyl were tested and worked well. However, Capto butyl offers best performance regarding endotoxin clearance.

### Ultrafiltration/Diafiltration

This step utilizes the same principle detailed in the section Tangential Flow Filtration step discussed above (the step after the clarification step). The aim is to adjust the pDNA concentration to a level that is compatible with the forthcoming enzymatic digestion used to linearize the plasmid. Typically, this concentration is about 0.5 mg DNA/mL. By using the TFF technique, it is possible to concentrate the product coming from the HIC step, commonly below the target concentration, to reach the desired concentration. The second purpose of this step is to change the buffer. The enzymatic digestion will work well in a 10 mM Tris buffer (pH 8.0), so it is required to remove the buffer derived from the HC FT and put the pDNA in this appropriate 10 mM Tris buffer. This is achieved dy UF/DF with a 10 to 12 DV diafiltration step.

Collection and/or Storage of Final Product

The pDNA should now be ready for use in further experiments and/or for production of RNA.

The pDNA is aliquoted in convenient volume of sample (typically 40 mL samples) and stored at −70° C. until further processing. The next step in the process is the enzymatic digestion at a unique restriction site in order to linearize the plasmid. The so linearized plasmid is finally purified (i.e. remove digestion enzyme) to be ready to be used for an in-vitro transcription (namely a chemical synthesis of RNA using oligonucleotides and RNA polymerase).

### Adjustments for Larger Scale Purification

Commercial kit purification processes by design are not scalable and pose a serious challenge to fulfil the high quantity and quality demands of pDNA for industrial applications, such as for preparation of pDNA to be used to prepare a biological product. Consequently, consideration has been given to providing a scalable purification process with commercial advantages. In fact, in a disclosed large-scale purification method, a downstream process for the purification of supercoiled plasmid DNA is described wherein one is able to scale up the process to achieve bulk pDNA at a high-performance level and select a fraction of supercoiled DNA which results in a purer pDNA product that was quicker and more cost effective to produce. The basic small-scale process disclosed herein can be scaled up to a large-scale process with surprising ease. In addition, the large-scale process has been optimized to achieve a large amount of purified pDNA, potentially with smaller volumes and smaller columns than required than typically needed or required to produce the same amount of purified pDNA by other large-scale processes. The optimized procedure yields pDNA that is more than 80% in the supercoiled form and virtually free from other unwanted isoforms.

An optimized larger scale pDNA purification process was performed in accordance with Examples 1-10 but with the following exceptions listed below.

The small-scale processes described in Examples 1-10 includes the purification of pDNA that encoded a SAM molecule having about 12-18 kbp whereas in the large-scale process described starting in Example 11, classical pDNA which is about 3-fold smaller, was employed, allowing for higher volumetric yield and broader applicability.

In Example 11, described below, downstream purification activities were utilized to achieve the large-scale purification of pDNA. More specifically, downstream processing methods coupled with anion-exchange chromatography aided in the separation of any unwanted plasmid variants that is ineffective in transferring genetic material and endotoxins (LPS) from the supercoiled plasmid that make up the desired purified pDNA product. Supercoiled pDNA, due to its structure and conformation extremely compact and functional, is considered the most efficient isoform at inducing gene expression as compared to these other conformational variants. Henceforth, cellular impurities must be removed during the downstream process to produce both the desired large quantity and high-quality yield of pDNA product that meets the internal quality standards as described in the aforementioned examples as well as that of the commercial market.

The Properties and/or Chemical Make of Purified pDNA Composition

The present invention also relates to a highly purified pDNA that has a large amount of pDNA in the supercoiled form and relatively small amounts of open-circular (or “nicked”) pDNA. The purified pDNA in the supercoiled form can be made by processes described in the Embodiments and Examples below or by other processes. The purified pDNA, in some embodiments, may have the following properties.

Amount of Supercoiled pDNA—In one embodiment, the amount of pDNA that is in the supercoiled form can be more than 80% and can be virtually free from other unwanted isoforms. The pDNA is preferably more than 85% in the supercoiled form, such as 85-99%, 85-96%, 85-95%, 87-95% or 84-94% in the supercoiled form.

Amount of Open Circular (OC) DNA—In one embodiment the amount of OC DNA (based on the total amount of pDNA) is below 25%, preferably less than 20%, more preferably less than 15%, most preferably less than 12%. For example, the amount of OC DNA can be between 5 and 23%, or between 5 and 22%, or between 5 and 21%, or between 5 and 15% or between 5 and 12% or between 8 to 21% or between 10 and 20%.

Ratio of scDNA to ocDNA—In some embodiments, the ratio of supercoiled plasmid DNA (scDNA) to non-supercoiled pDNA (non-scDNA, such as open circular plasmid DNA (ocDNA)) can be maximized or increased by one or more process steps that separate or allow for separation of scDNA and ocDNA or process steps that increase the amount of scDNA to ocDNA. For example, the ratio of scDNA to ocDNA can be at least 5, preferably at least 6 and more preferably at least 7.

Amount of Other Conformations—The amount (%) of other conformation (other than scDNA and ocDNA) can be reduced to very low levels such as below 5%, below 4%, below 3%, below 2% or below 1%, below 0.5%, below 0.3%, below 0.2% or below 0.1%. In this regard, as reported in Table 12 below the amount of other conformations can be reduced to levels that are undetectable (0%) by FEMTO-Pulse.

A260/A280 Value—The ratio of absorbance at 260 and 280 nm is used to assess the purity of DNA. According to the present invention, the value for pure DNA is at least 1.8, preferably between 1.8 and 2.0, and more preferably between 1.9 and 2.0.

Total Protein Content—The total protein content is determined as a ratio of μg of Protein (measured with a total protein assay) per 100 μg of DNA (measured by Nanodrop). According to the present invention, μg of Protein/100 μg of DNA=%. The protein content therefore is preferably less than 5%, more preferably less than 2%, even more preferably less than 1.0%. Small amounts of residual protein may be present, such as amounts of 0.4% or more, or 0.5% or more or 0.6% or more or 0.6 to 0.7%.

Residual Entotoxin Concentration—In the final product, the amount of residual endotoxin relative to the quantity of pDNA is usually less than less than 1,500 EU/mg, preferably less than 300 EU/mg, such as 200-300 or 225-300 EU/mg, more preferably less than 100 EU/mg, even more preferably less than 50 EU/mg and most preferable less than 20 EU/mg.

Amount of Residual RNA—The amount of residual RNA is calculated using the following formula: [RNA]μg/ml/([DNA]μg/ml+[RNA]μg/ml)*100=%. According to the present invention, the amount of residual RNA is less than 8%, preferably less than 6%, more preferably between 2 and 8% and most preferably between 4 and 6%.

Compositions Containing Ammonium Sulphate—In one embodiment, the plasmid composition can be a liquid composition containing the purified pDNA (as described in the other paragraphs in this section) and further containing ammonium sulphate at a molar concentration of 1.7M, preferably 1.5 to 1.85 M. This composition can be produced as one or more steps of the process of the present invention once the desired purity level is reached, particularly at the end of the HIC chromatography step. Of note, the HIC step is an intermediate step in the process, therefore the final UF/DF should be made to get the pDNA in appropriate buffer, unless it is likely that the ammonium sulphate will interfere in further IVT process reaction.

### Embodiments of the Invention

Various embodiments of the invention include, without limitation,

In some aspects, the present invention is directed to the following embodiments.

1. A method of purifying plasmid DNA (pDNA), comprising the steps of:
subjecting a sample comprising pDNA to a core bead flow-through chromatography step to reduce the level of at least endotoxin to produce a core bead flow-through; and
subjecting the core bead flow-through to an anion exchange chromatography step.

2. The method of embodiment 1, wherein the core bead flow-through chromatography removes materials by both size exclusion and binding properties.

3. The method of embodiment 1 or 2, wherein the core bead flow-through chromatography is performed with beads that have an inactive shell containing pores and a core underneath the inactive shell, wherein core ligands located in the core are in fluid communication with the exterior of the beads through said pores.

4. The method of embodiment 3, wherein said core ligands are both hydrophobic and positively charged.

5. The method of embodiment 2, 3 or 4, wherein the core bead flow-through chromatography is performed using beads comprising a shell containing pores and a core underneath the shell, wherein the pores in the shell have a cut-off at a molecular mass (Mr) of 400 kd or greater and exclude materials having a Mr greater than the cut-off.

6. The method of embodiment 2, 3 or 4, wherein the core bead flow-through chromatography is performed using beads comprising a shell containing pores and a core underneath the shell, wherein the pores in the shell have a cut-off at a molecular mass (Mr) of 600 kd or greater and therefore exclude materials having a Mr greater than the cut-off.

7. The method of embodiment 2, 3 or 4, wherein the core bead flow-through chromatography is performed using beads comprising a shell containing pores and a core underneath the shell, wherein the pores in the shell have a cut-off at a molecular mass (Mr) of 700 kd or greater and therefore exclude materials having a Mr greater than the cut-off.

8. The method of any of the preceding embodiments, wherein a buffer containing sodium chloride is used in the core bead flow-through chromatography step.

9. The method of any of embodiments 1-7, wherein a buffer containing sodium chloride is used in the anion exchange chromatography step.

10. The method of any of embodiments 1-7, wherein a buffer containing sodium chloride is used in the core bead flow-through chromatography step and the concentration of the sodium chloride in the anion exchange chromatography step.

11. The method of embodiment 10, wherein the concentration of sodium chloride in the buffer used in the core bead flow-through chromatography step and the anion exchange chromatography step are both in the range of 150 mM to 900 mM.

12. The method of any of the preceding embodiments, wherein the sample comprising pDNA is obtained from E. coli cells that have been lysed by alkaline lysis.

13. The method of embodiment 1, wherein said pDNA encodes a mRNA.

14. The method of embodiment 1, wherein said pDNA encodes a mRNA of greater than 5,000 bases.

15. The method of embodiment 1, wherein said pDNA encodes a SAM molecule.

16. The method of embodiment 1, which is a large-scale batch purification method.

17. A method of improving the quality of a template pDNA prior to an in vitro transcription reaction, comprising the steps of: (i) subjecting a sample comprising pDNA to a core bead flow-through chromatography step to reduce the level of at least endotoxin to produce a core bead flow-through; (ii) subjecting the core bead flow-through to an anion exchange chromatography step; and (iii) collecting the fraction comprising super coiled (sc) pDNA

18. A method for purifying pDNA comprising the steps of: i) lysing a large sample of host cells in a large buffer volume to obtain a cell lysate and treating with a salt to precipitate the RNA to produce a neutralized cell lysate; ii) producing a clarified cell lysate by clarifying the neutralized cell lysate in the appropriate excipient buffer solution and filtering the clarified cell lysate through tangential flow filtration to produce a filtered pDNA sample; iii) subjecting the filtered pDNA sample to a core bead flow-through chromatography step to produce a core bead flow-through; iv) subjecting the core bead flow-through to an anion exchange chromatography step wherein different plasmid DNA isoforms are separated into fractions, and a desired scDNA fraction or fractions are eluted; v) further removing endotoxin impurities from desired scDNA fraction or fractions by subjecting said fraction or fractions to a chromatography step utilizing an HIC resin on a Captobutyl column to produce a Captobutyl eluate; and vi) subjecting said Captobutyl eluate to a second tangential flow filtration step to filter and concentrate the Captobutyl eluate to produce purified pDNA in a form suitable for storage.

19. The method of embodiment 18, wherein said pDNA purification method comprises at least 2, preferably 3, chromatography steps to achieve a large-scale batch of high-purity pDNA product.

20. The method of embodiment 18, wherein in said step (i) the lysing step comprises use of an alkali salt and an ionic detergent.

21. The method of embodiment 18, wherein in said step (i) the lysing step comprises agitating the cell lysate in a stirred tank to achieve high-purity pDNA homogeneity.

22. The method of embodiment 18, wherein in said step (v) the scDNA product is spiked with ammonium sulphate.

23. The method of embodiment 18, wherein in said step (i) the volume of said culture is at least 15 liters.

24. The method of embodiment 18, wherein in said step (ii), the excipient is CaCl₂).

25. A plasmid DNA composition comprising pDNA wherein at least 80% of the plasmid DNA is in supercoiled form, less than 15% is in open-circular form and less than 5% is in other isoforms, all percentages being based on the total amount of pDNA present.

26. The plasmid DNA composition according to embodiment 25 comprising no more than 15% nicked pDNA during separation of different plasmid isoforms present in the clarified lysate.

27. The use of pDNA produced according to the method of embodiment 1 or 18 or the plasmid of embodiment 25, in an in vitro transcription reaction to synthesize RNA.

28. The pDNA produced according to the method of embodiment 1 or 18, wherein said pDNA suitable for pharmaceutical use.

In other aspects, the present invention is based on the following embodiments:

1. A method of purifying plasmid DNA (pDNA), comprising the steps of:
subjecting a sample comprising pDNA to a core bead flow-through chromatography step to reduce the level of at least endotoxin to produce a core bead flow-through; and
subjecting the core bead flow-through to an anion exchange chromatography step.

2. The method of embodiment 1, wherein the core bead flow-through chromatography removes materials by both size exclusion and binding properties.

3. The method of embodiment 1 or 2, wherein the core bead flow-through chromatography is performed with beads that have an inactive shell containing pores and a core underneath the inactive shell, wherein core ligands located in the core are in fluid communication with the exterior of the beads through said pores.

4. The method of embodiment 3, wherein said core ligands are both hydrophobic and positively charged.

5. The method of embodiment 2, 3 or 4, wherein the core bead flow-through chromatography is performed using beads comprising a shell containing pores and a core underneath the shell, wherein the pores in the shell have a cut-off at a molecular mass (Mr) of 400 kd or greater and therefore exclude materials having a Mr greater than the cut-off 6. The method of embodiment 2, 3 or 4, wherein the core bead flow-through chromatography is performed using beads comprising a shell containing pores and a core underneath the shell, wherein the pores in the shell have a cut-off at a molecular mass (Mr) of 600 kd or greater and therefore exclude materials having a Mr greater than the cut-off.

7. The method of embodiment 2, 3 or 4, wherein the core bead flow-through chromatography is performed using beads comprising a shell containing pores and a core underneath the shell, wherein the pores in the shell have a cut-off at a molecular mass (Mr) of 700 kd or greater and therefore exclude materials having a Mr greater than the cut-off.

8. The method of any of the preceding embodiments, wherein a buffer containing sodium chloride is used in the core bead flow-through chromatography step.

9. The method of any of embodiments 1-7, wherein a buffer containing sodium chloride is used in the anion exchange chromatography step.

10. The method of any of embodiments 1-7, wherein a buffer containing sodium chloride is used in the core bead flow-through chromatography step and the anion exchange chromatography step.

11. The method of embodiment 10, wherein the concentration of sodium chloride in the buffer used in the core bead flow-through chromatography step and the anion exchange chromatography step are both in the range of 150 mM to 900 mM.

12. The method of any of the preceding embodiments, wherein the sample comprising pDNA is obtained from E. coli cells that have been lysed by alkaline lysis.

13. The method of embodiment 1, wherein said pDNA encodes a mRNA.

14. The method of embodiment 1, wherein said pDNA encodes a mRNA of greater than 5,000 bases.

15. The method of embodiment 1, wherein said pDNA encodes a SAM molecule.

16. The method of embodiment 1, wherein said pDNA encodes a SAM molecule encoding a Hepatitis antigen.

17. The method of embodiment 1, wherein said pDNA encodes a SAM molecule encoding COVID-19 spike protein.

18. The method of embodiment 1, wherein said pDNA encodes a SAM molecule encoding a rabies antigen.

19. The method of embodiment 1, wherein said pDNA encodes a SAM molecule encoding a HSVth antigen.

20. A method of improving the quality of a template pDNA prior to an in vitro transcription reaction, comprising the steps of: (i) subjecting a sample comprising pDNA to a core bead flow-through chromatography step to reduce the level of at least endotoxin to produce a core bead flow-through; (ii) subjecting the core bead flow-through to an anion exchange chromatography step; and (iii) collecting the fraction comprising super coiled (sc) pDNA.

### EXAMPLES

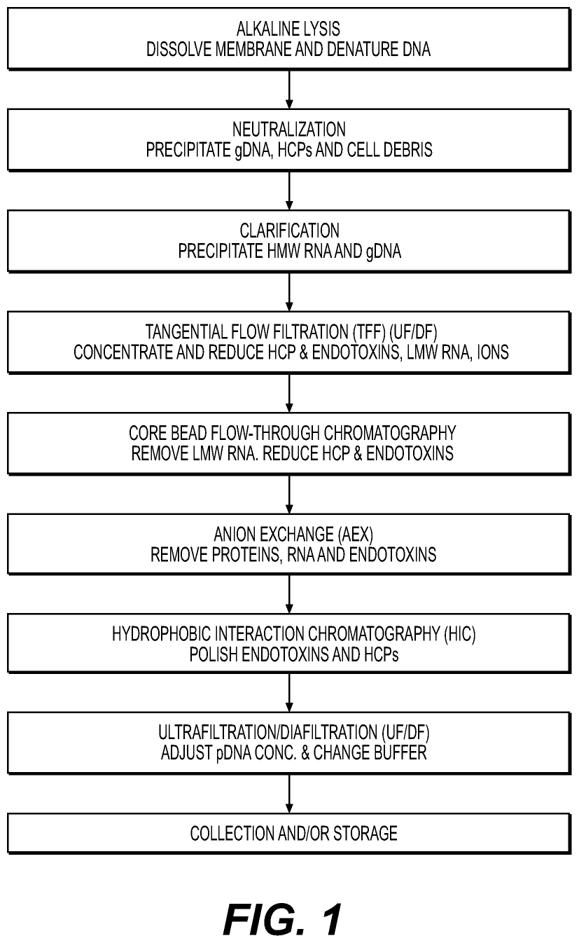

An overview of the process of the present invention is shown in FIG. 1. To prepare a single batch of material using the process, the process can be conducted over a period of three days; however, the exact number of hours or days can be varied based on a number of factors including, without limitation, batch size and equipment size. Details of processing conditions used in Examples 1-10 are summarized in the following Tables 1-3.

TABLE 1
(Day 1)

| | |
|---|---|
| Cell Lysis | Cells Alkaline Lysis |
| and | Resuspend 50 g of E. coli paste (equivalent of 1 L harvest |
| Neutral- | collected) in 500 mL of 150 mM Tris - 10 mM EDTA - |
| ization | 50 mM Glucose - pH 8.0 |
| | Add 500 mL of 0.2M NaOH - 1% SDS - Check pH (12.0 ± |
| | 0.5) - Agitate very smoothly during 5 min |
| | Neutralize by adding progressively ~120 mL of cold 3M |
| | potassium acetate (pH 5.5) to reach a pH between |
| | 7.0-8.0 - Leave on ice for 15 min |
| | Adjust pH to 7.8 ± 0.1 with 1.8M formic acid or NaOH 0.5M |
| Clarifi- | Precipitation: Add 2M CaCl₂ to reach a final concentration |
| cation | of 0.4M CaCl₂ - incubate for 15 min |
| | Centrifugation: 45 min at 13 000 × g - 4° C. - collect |
| | supernatant |
| | Filtration of the lysate SN through 0.45/0.2 μm Sartobran P |
| | filter |
| TFF | Pellicon 3 - 0.11 m² - Biomax 50 kDa |
| | Concentration: Concentrate filtered SN about 11× to reduce |
| | volume (1300 ml → 120 ml) |
| | Diafiltration 1: diafilter 20 DV against 10 mM Tris - pH 7.5 |
| | Diafiltration 2: diafilter 10 DV against 10 mM Tris - 10 mM |
| | EDTA - pH 7.5 |
| | Collect retentate and rinse the system with 10 mM Tris - |
| | 10 mM EDTA - pH 7.5 |
| | Pool retentate and rinse fraction: final Fcc = 8.5x |

TABLE 2
(Day 2)

| | |
|---|---|
| Multimodal | Captocore 700 (FT MODE) - CV = 53 mL |
| Xto | Sample conditioning: UF retentate is spiked to 0.5M NaCl |
| | with 5M NaCl stock solution |
| | Equilibration: 8 CV of 10 mM Tris - 10 mM EDTA - 0.5M |
| | NaCl - pH 7.5 |
| | Load: Total spiked UF-R (~3 CV) - collect FT |
| | Wash: 5 CV of 10 mM Tris - 10 mM EDTA - 0.5M NaCl - pH 7.5 - |
| | collect 1^{st} CV with FT |
| | Regeneration: 6 CV of 1M NaOH - 30% isopropanol including a static |
| | pause of 1 h after 3 CV |
| AEX | Fractogel EMD DMAE (BIND-ELUTE MODE) - CV = 30 mL |
| Xto | Equilibration: 4 CV of 25 mM Tris - 1 mM EDTA - 0.5M NaCl - pH 7.5 |
| | Load: Total of the Captocore FT (~5 CV) |
| | Wash 1: 4 CV of 25 mM Tris - 1 mM EDTA - 0.5M NaCl - pH 7.5 |
| | Wash 2: 3 CV of 25 mM Tris - 1 mM EDTA - 0.65M NaCl - pH 7.5 |
| | Elution Gradient: from A: 25 mM Tris - 1 mM EDTA - 0.65M NaCl pH |
| | 7.5 to B: 25 mM Tris - 1 mM EDTA - 1.0M NaCl - pH 7.5 on 15 CV: |
| | collect 2^{nd} peak or 2^{nd} part of the peak |
| | Regeneration: 5 CV of 0.5M NaOH - 2M NaCl |

TABLE 3
(Day 3)

| | |
|---|---|
| HIC | Captobutyl (FT MODE) - CV = 30 mL |
| Xto | Sample conditioning: DMAE eluate is spiked to 1.7M (NH₄)₂SO₄ with 5M |
| | NaCl stock solution |
| | Equilibration: 5 CV of 10 mM Tris - 1 mM EDTA - 3M NaCl - pH 7.5 |
| | Load: Total of spiked DMAE eluate (~6.5 CV) - collect FT |
| | Wash 1: 5 CV of 10 mM Tris - 1 mM EDTA - 3M NaCl - pH 7.5 - collect 1^{st} |
| | CV with FT |
| | Wash 2: 6 CV of 10 mM Tris - 1 mM EDTA - pH 7.5 |
| | Regeneration: 5 CV of 10 mM Tris - 50% Ethylene glycol |
| TFF | Pellicon 3 - 88 cm² - Biomax 30 kDa |
| | Concentration: Concentrate the Catpobutyl FT to reach a concentration of |
| | 0.55 mg/ml (monitoring with Nanodrop) |
| | Diafiltration: diafilter 10 DV against 10 mM Tris - pH 8.0 |
| | Collect retentate → purified plasmid fraction (pDNA) |
| Storage | Storage of pDNA at −20° C. |

Unless otherwise noted, E. coli cells harboring plasmid DNA that encodes a SAM molecule were used as a source of the plasmids in the following Examples. The SAM molecule that was initially tested is designed to express a COVID-19 spike protein when introduced into a subject receiving a vaccine containing the SAM molecule. The development was initially made using pDNA encoding COVID-19 spike protein (about 25% of the development work) and thereafter the majority of the development work (about 75% of the development work) was done using a SAM plasmid designed to express a Hepatitis viral antigen. Some additional work was also done with other plasmids, as reported below. Experiments E1 to E14 (#1 to #14) were done using SAM-COVID-19 construct. Experiments E15 to E44 (#15 to 44) were done using a SAM Hepatitis viral antigen construct. In Experiments E45 (#45) and higher, different constructs were evaluated (HV, COVID-19, Rabies and HSVth). Thus, except for a few designated experiments in Example 5 (run 18-b in Table 5 which was done with COVID-19 pDNA) below, all of the work reported in Examples 1-9 was done with pDNA encoding a Hepatitis antigen. E11 was done with SAM-COVID. The pDNA plasmids tested in Example 10 were also prepared in accordance with the procedures reported in Examples 1-9. Most of the experiments were generated using pDNA encoding a protein for treating Hepatitis Virus (HV) infection. This protein is also referred to as Hepatitis antigen. Six batches of pDNA production are reported in this application.

These six batches utilized four pDNAs as follows:
E40 (Hepatitis antigen)
E43 (Hepatitis antigen)
E45 (COVID)
E46 (Rabies)
E47 (HSVth)
E52 (COVID)

Purified pDNA produced in accordance with these Examples can be used to make SAM molecules by, for example, in vitro transcription (IVT) or other techniques.

### Example 1—Cell Lysis and Neutralization

E. coli cells were lysed and neutralized using the conditions reported in Table 1 to prepare a neutralized cell lysate. This Example corresponds to the first two boxes in the flow chart shown in FIG. 1 (alkaline lysis and neutralization).

### Example 2—Clarification

Neutralized cell lysate obtained in accordance with Example 1 was clarified by precipitation, centrifugation and filtration using the conditions reported in Table 1 to prepare a clarified composition. In this clarification step, CaCl₂) is added directly to the neutralized cell lysate of Example 1 to bring the CaCl₂) concentration to 0.3 M and a single centrifugation step is employed at a centrifugation speed of 13,000 g for 45 minutes. In some processes, two centrifugation steps are needed (one before and another one after CaCl₂) addition) at this stage and the single centrifugation step employed in this Example simplifies the process.

### Example 3—Tangential Flow Filtration

Tangential Flow Filtration (TFF) was performed on the clarified composition prepared in Example 2. TFF reduces the volume of the clarified composition while retaining pDNA in the composition. TFF also allows various molecules of small sizes to pass through the membrane in the TFF system so that they are separated from the pDNA, thus reducing the concentration of these small molecules. The TFF also makes it possible to reduce the concentration of various undesired ions by buffer exchange using diafiltration to make the product of this step more suitable for further downstream processing. The TFF was conducted under the conditions shown in Table 1 above. The product of this TFF step is a retentate that retains most or all of the pDNA in a reduced volume of liquid and that contains reduced relative amounts of some undesired impurities.

### Example 4—Core Bead Flow-Through Chromatography (Hepatitis Antigen pDNA)

Core Bead Flow-Through Chromatography was performed to separate undesired small molecules such as LMW RNA from the pDNA. This step also reduces the concentration of HCP and endotoxins. This is accomplished in this Example by using resin beads that have surface pores that allow molecules below a certain size to pass into the resin beads. Once the small molecules have passed through the surface pores that are present in the surface of the resin beads, they encounter ligands that retain the small molecules inside the pores. These interior binding ligands are not on the exterior of the beads. The interior of the beads may have two or more different types of ligands with different binding/retaining specificity. The desired pDNA is too large to pass through the pores and into the interior of the beads and thus the pDNA passes through the TFF system or TFF column as a core bead chromatography “pass through”. Such Core Bead Flow-Through Chromatography is sometimes referred to as multimodal chromatography because it separates material based on two distinct properties: size and binding properties. In this Example, the beads (chromatography medium) that were used to separate materials based on size and binding properties were CaptoCore 700 manufactured by Cytiva. Capto Core 700 beads have octlyamine ligands inside of the beads which binds smaller proteins and impurities that enter into the core of the particles, due to both charged and hydrophobic interactions. The material that passes through the column in this step is referred to as a “flow through” (FT).

CaptoCore 700 was tested in the flow through mode under the general conditions shown in Table 2 above. This step was performed at various different NaCl concentrations in order to assess its ability to remove various impurities. NaCl was found to be more effective than the sample control (E35a-ctrl) that did not contain NaCl (0 M NaCl as shown in the following Table 4:

TABLE 4
(SAM-Hepatitis antigen Construct)

| | | | | % Protein | Residual |
|---|---|---|---|---|---|
| | | | | μg Prot/100 | Endotoxin |
| | | pDNA | Residual | μg DNA | EU/mg DNA |
| | Salt | Yield | RNA | (clearance | (clearance |
| Fraction | Condition | % | % | factor x) | factor x) |
| CaptoLoad | 0M NaCl | | 75.1 | 1364 | 2465 |
| E35a-FT (ctrl) | 0M NaCl | 61.7 | 13.2 | 71.1 (19x) | 354.4 (7x) |
| E35b-FT | 0.35M NaCl | 80.8 | 12.6 | 18.2 (75x) | 33.4 (74x) |
| E35c-FT | 0.50M NaCl | 69.8 | 13.7 | 20.7 (66x) | 27.4 (90x) |
| E35d-FT | 0.75M NaCl | 78.8 | 13.2 | 17.4 (78x) | 22.3 (110x) |

The following effects of NaCl concentration on CaptoCore were observed:
No loss of pDNA (yield acceptable)
No loss of efficacy to clear RNA (compared to Control)
3 to 4 times more efficient to remove proteins
10 to 15 times more efficient on Endotoxin clearance

Considering the above, an ideal NaCl concentration is 0.5M, which is the same NaCl concentration used for the DMAE load in the following AEX step. Using the same NaCl concentration in this step as used for the DMAE load in the following anion exchange step (Example 5) results in process simplification.

### Example 5—Anion Exchange (AEX) (SAM—Hepatitis Antigen Construct)

The pass through from Example 4 was then subjected to Anion Exchange in this Example. The purpose of this step is to remove (or reduce the amount of) proteins, RNA and endotoxins from the flow through from the previous step. In addition, this step separates the following two isoforms of plasmids: (1) supercoiled (SC) plasmids and (2) open circular (OC) plasmids. The AEX resin used in this Example was Fractogel EMD DMAE in the bind-elute mode. The details of the AEX process are described in Table 2 above. The first screening of AEX (E11) was done using SAM-COVID construct while later experiments on AEX (E18 and E33) were done using a SAM Hepatitis antigen construct.

Various AEX resins were screened for their effectiveness in polishing at this step. The results of these experiments are reported in Table 5.

TABLE 5
Screening AEX Resin for Polishing
(COVID-19 and SAM Hepatitis antigen Constructs)

| | | % Yield | % RNA | % Protein | Endotoxin |
|---|---|---|---|---|---|
| Run | Fraction | DNA | (Qubit) | (Nanodrop) | Reduction (x) |
| 11 | AEX Load | — | 8.3 | 16.5 | — |
| 11-1 | Eluate Praesto Q | 64.0 | 5.5 | 0.8 | 3x |
| 11-5 | Eluate EMD DEAE | 71.0 | 5.0 | 1.2 | 3x |
| 11-6 | Eluate TMAE HiCap | 26.2 | 7.1 | 1.7 | 1x |
| 18-b | Eluate EMD DMAE | 87.6 | 3.6 | 0.9 | 9x |

### DMAE Performance:

% RNA reduction: 2×
% Protein reduction: 18×
Endotoxin level reduction: 9×
Experiment E11 (1-6) was performed using SAM COVID material. Experiment E18 (a-b) was done using SAM Hepatitis viral antigen material. This Table is a summary of relevant data.

EMD DMAE (which is a type of Fractogel) was selected as a preferred candidate from the above four AEX materials based on a combination of high yield of DNA (87.6%) and a small amount of residual protein 0.9%). After selecting Fractogel EMD DMAE as the preferred material, the following experiments were performed using the conditions reported in Table 2 above.

The elution gradient results for the Fractogel EMD DMAE (Run 18-b in Table 5) was performed with an elution gradient of 0.6 M-1.2 M NaCl.—Fractogel EMD DMAE on a YMC 10/5 column. The column has a diameter of 10 mm, height of 5 cm, and a CV of 3.9 mL.

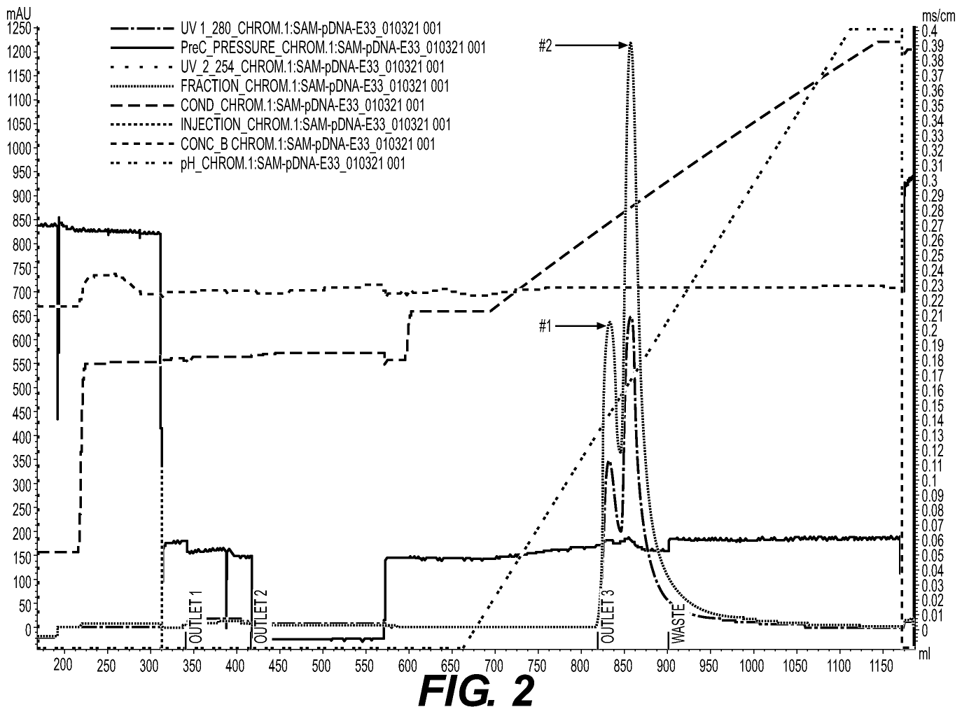

The chromatogram shown in FIG. 2 is coming from run E33 using a SAM Hepatitis antigen construct. The E33 run is performed with a 0.5M NaCl spiking and a 0.6-1.2M NaCl for elution gradient but using a Hi16/h=15 cm (CV=30 mL) column size.

The run E18b (screening purpose) was made in same buffer conditions but on a smaller column (h=5 cm/VC=3.9 mL) which has not sufficient resolution to show 2 peaks (we can only see a shoulder+main peak).

The following Table 6 provides process optimization results for the Fractogel EMD DMAE (run E33) using a Hepatitis antigen construct.

The below Table 7 shows the results of a gradient of 0.6-1.2M NaCl on Fractogel EMD DMAE.

TABLE 7
(SAM Hepatitis antigen Construct)

| | | | Load Q |
|---|---|---|---|
| Lane | Run # | Sample | (μg) |
| 1 | Unstained | MW | — |
| 2 | SAM-pDNA-E33 | DMAE Load | 0.5 |
| 3 | | DMAE Elution 1 | 0.5 |
| | | (25% of material) | |
| 4 | | DMAE Elution 2 | 0.5 |
| | | (75% of material) | |

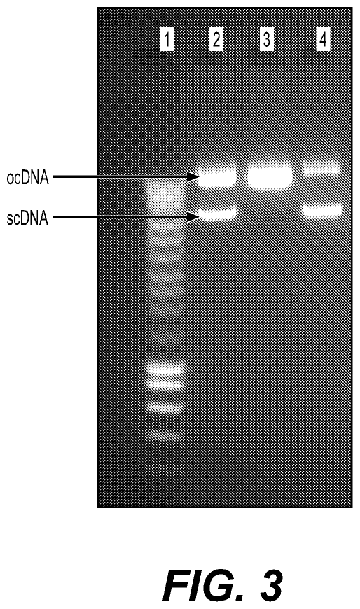

FIG. 3 corresponds to the experiment reported in the above Table 7 (using the SAM Hepatitis antigen construct) and shows that peak #1 is almost exclusively ocDNA (lane 3) and that peak #2 is mainly scDNA (lane 4). DMAE is therefore able to separate 2 plasmid forms (if required). An optimized NaCl gradient of 0.65-1.0 NaCl may be more convenient to improve resolution.

### Example 6—AEX Optimization (Hepatitis Antigen pDNA)

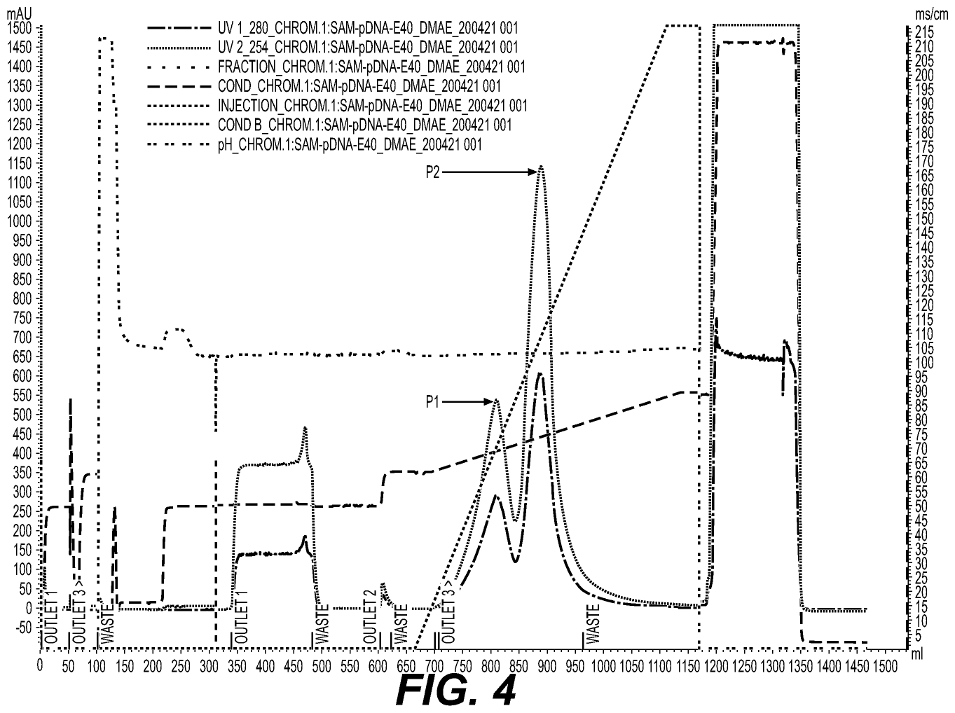

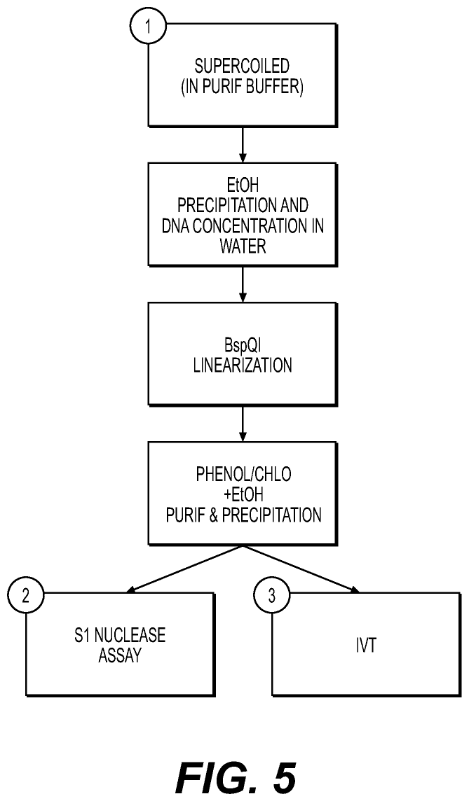

FIGS. 4-6 relate to HVpDNA. The importance of the separation of scDNA from ocDNA is illustrated in the following Figures. The chromatogram in FIG. 4 displays 2 peaks in elution profile (named p1 and p2 in sequence of elution). Each peak has been individually collected and analysed according the schema in FIG. 5. Namely, each fraction has been precipitated with ethanol, re-suspended in water and digested by BspQ1 restriction enzyme to enable linearization. Once linearized, a S1 nuclease assay was performed (which cut the double stranded DNA if one of the 2 strand is already cut=nicked) and also an In Vitro Transcription into RNA to check RNA quality issued from both forms of plasmids. FIG. 6B (bottom part) displays a more diffuse band (smear) for the ocDNA fraction of 2 different lots [E36 & E40] while the scDNA fraction show an intact band. This tends to demonstrate that ocDNA contains much more nicks than scDNA. However, it did not result in a large difference in RNA quality after IVT (FIG. 6C), perhaps because the RNA polymerase is able to “correct” the cut in the DNA strand by passing over it.

FIG. 4 is a DMAE chromatogram showing the two peaks at elution. This sample is pDNA encoding Hepatitis antigen. This FIG. 4 was generated using the same conditions as used to generate the chromatogram in FIG. 2, with the following exception. As explained above, the gradient used in E40 (FIG. 4) is 0.65-1.0 M NaCl, while for E33 (FIG. 2) it is still 0.6-1.2 M. This is due to gradient optimization between E33 and E40. Like FIG. 2, Peak 1 is mainly ocDNA and Peak 2 is mainly scDNA. In FIG. 2, the right axis is labeled “mS/cm” which means milliSiemens/cm (a unit of conductivity).

FIG. 5 is a flow chart that summarizes further treatment of the scDNA obtained from AEX purification of the HV material described above.

FIGS. 6A-6D report further experiments that characterize the Hepatitis plasmid at various stages of the further treatment steps shown in FIG. 5. In FIGS. 6A-6D, FIG. 6A is plasmid DNA (scDNA and ocDNA), FIG. 6B is linearized DNA (after digestion) and FIG. 6C is RNA (after IVT from linearized DNA). The results are explained below as follows:

Sample 12—E25 UF: UF-Retentate (material recovered from UF step)

Sample 13—E36 PIC 1-peak 1 of DMAE chromato elution of run E36 (ocDNA fraction)

Sample 14—E36, PIC 2: peak 2 of DMAE chromato elution of run E36 (scDNA fraction)

Sample 15—E40, PIC 1: peak 1 of DMAE chromato elution of run E40 (ocDNA fraction)

Sample 16—E40, PIC 2: peak 2 of DMAE chromato elution of run E40 (scDNA fraction)

FIG. 6A is a gel that shows the materials in the box labeled “1” in FIG. 5.

FIG. 6B is a gel that shows the materials in the box labeled “2” in FIG. 5. The bands in the upper part of FIG. 6B are before the S1 nuclease assay and the bands in the lower part of the gel are after the S1 nuclease assay. Lane 2 and 4 (pic1) are displaying a diffuse band (smear): that is evidence that there are linear pDNA of many different sizes (thus migrating on different positions). If there are different sizes, it means there the pDNA is nicked and the S1 assay has degraded the original DNA nicked into smaller DNA (incomplete sequence). Thus, PIC1 lanes are not the desired form, while PIC2 lanes are desired plasmid forms (intact/complete linear DNA without nick).

FIG. 6C is a gel that shows the materials in box labeled “3” in FIG. 5, which is after IVT synthesis. IVT was carried out reaction Master Mix (42 mM Tris.HCl pH 8.0, 25.3 mM MgCl2, 6.3 mM each NTP, 10.5 mM DTT, 2.1 mM spermidine, 52.6 ng/μL linearized sample DNA, 0.002 U/μL yeast inorganic pyrophosphatase (NEB), 1.05 U/μL Rnase Inhibitor (NEB)). Reactions were initiated by the addition of 50 nL of desalted, purified enzyme followed by incubation at 30° C. for 2 hours.

FIG. 6D shows concentration of Hepatitis antigen RNA production after IVT by sample for the samples reported in FIGS. 6A-6C (reported in ng/μl). These are the derived concentrations of RNA after IVT, based on scanning densitometry of the results in FIG. 6C. FIG. 6D suggests the same thing as FIG. 6C, i.e., no major difference was observed in RNA concentration after IVT whether they come from PIC1 (ocDNA) or PIC2 (scDNA). The % integrity of the RNA produced from the IVT was analyzed. “% RNA integrity” refers to the percent of RNA molecules that are full-length RNA consisting of entire sequence encoded in DNA template to the total RNA, e.g., have both the 5′ and 3′ ends. % Purity can be determined using different techniques known to a skilled person, e.g., by RT ddPCR carried out according to the manufacture's recommendations. The assay utilizes two sets of primers and internal probes on the most terminal positions (i.e., 5′ and 3′ ends) of the full-length RNA. The presence of signal from both ends indicates a full-length RNA, the presence of only one signal indicates an incomplete product. The results of the ddPCR analysis on the two samples supports a conclusion that there is more full-length RNA after IVT for the PIC2 sample than for the PIC1 sample.

### Example 7—HIC (Hepatitis Antigen pDNA)

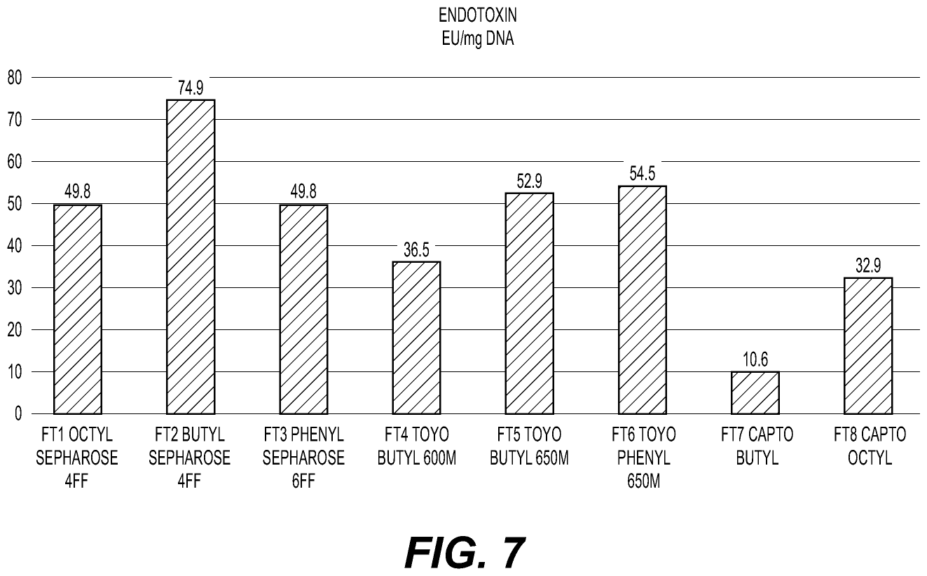

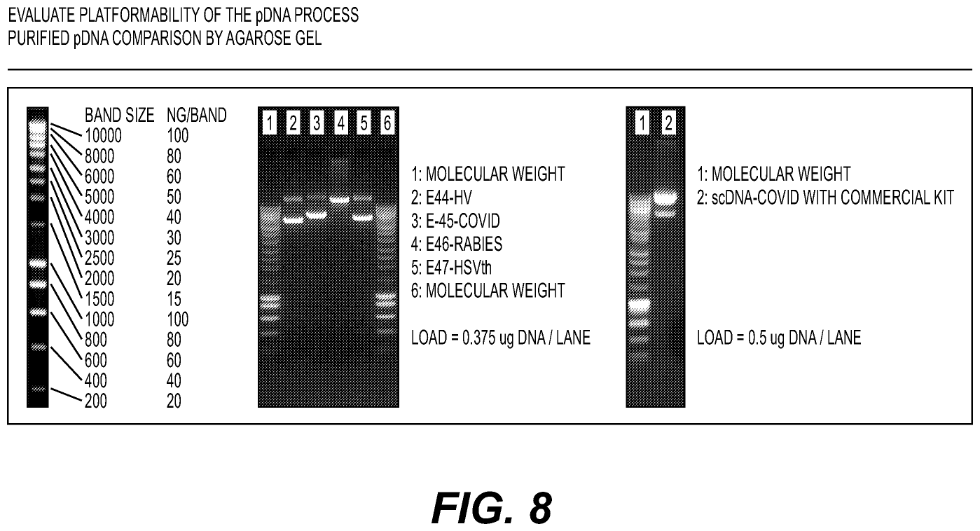

This step further reduces the level of LMW RNA, HCP and endotoxin impurities in the material obtained in peak 2 from Example 5. The following resins were screened for their ability to remove these materials: (1) Octyl Sepharose 4FF, (2) Butyl Sepharose 4FF, (3) Phenyl Sepharose 6FF, (4) Toyo Butyl 600M, (5) Toyo Butyl 650M, (6) Toyo Phenyl 650 Mm, (7) Capto Butyl, and (8) Capto Octyl. The ability of various resins to remove endotoxin are shown in FIG. 7. The results in FIG. 7 are from a screening of different HIC using a TECAN (High Throughput equipment). Captocore FT samples (from run E17) were used and spiked with 1.5M ammonium sulphate.

Equilibrate and chasing buffer=10 mM Tris−1.5M ammonium sulphate.

2 ml of spiked sample was loaded on a 200 μl column. The FT was collected from the column (like done with Captocore) and the HIC-FT was analysed for pDNA, RNA, protein and endotoxin content

For all of these screened resins, the following results were obtained: global pDNA yield was 85-90%, Residual RNA was less than 2%, protein content was 4-5% residual protein. However, as shown in FIG. 7. Capto Butyl was by far the best for endotoxin removal of endotoxin where the load of endotoxin was 4447 EU/mg of DNA. The following results were obtained for Capto Butyl: Yield pDNA=87%, % RNA less than 1%, % protein 4.4% and endotoxin=10.6 EU/mg DNA.

After selecting Captobutyl as the best resin, this step was performed using the conditions reported in Table 3 above.

The following experiment describes the effects of NaCl or (NH₄)₂SO₄ on CaptoButyl after DMAE.

TABLE 8
(Hepatitis antigen pDNA)

| | | pDNA | Residual | Residual | Residual | Endotox |
|---|---|---|---|---|---|---|
| | Salt | Yield | RNA | Protein | Endotoxin | clearance |
| Fraction | Condition | % | % | % | EU/mg | factor |
| E34 HIC | 1M NaCl | | 7.3 | 6.0 | 2403 | |
| Load | | | | | | |
| E34a-FT | 1.5M NaCl | 56.8 | 6.8 | 5.3 | 269 | 8.9 |
| E34b-FT | 2.25M NaCl | 68.2 | 5.7 | 4.9 | 250 | 9.6 |
| E34c-FT | 3M NaCl | 82.8 | 2.0 | 5.2 | 275 | 8.7 |
| E34d-FT | 0.85M NaCl + | 53.5 | 4.4 | 6.6 | 1342 | 1.8 |
| | 1M (NH4)2SO4 | | | | | |

For the experiments reported in Table 8, the DMAE eluates of runs E31 and E28 (both peak 1 and peak 2 to have physically enough material to make the experiment) were pooled. The pool was split into four parts and spiked with salts as shown below:
A: spike to 1.5M NaCl
B: spike to 2.25M NaCl
C: spike to 3.0M NaCl
D: spike to 1.0M Ammonium Sulphate

Note: Due to NaCl already present due to DMAE elution, it was considered that the starting concentration of NaCl [NaCl] was 0.9M for the spiking calculation. pDNA and contaminant content were measured in the HIC-FT of the 4 conditions (a/b/c/d). This was performed on prepacked HiScreen Captobutyl columns.

As shown in the above Table 8, 3M NaCl provides the best results, i.e., it provides the best pDNA yield, best residual RNA and best Endotoxin clearance factor (Endotoxin Load/Residual Endotoxin), with no impact on protein clearance. Use of NaCl alone is also easier to manage in that there is no mixing of salts (ammonium sulphate & NaCl have complex interactions) and NaCl is environmentally friendly, i.e., ammonium sulphate is a harmful waste.

More recent experiments in a larger scale pDNA purification process however have shown that ammonium sulphate alone at a particular molar concentration was more efficient at endotoxin clearance than NaCl, resulting in the same or better quality of highly purified pDNA. Accordingly, optimized conditions of this process and a large-scale process will be further discussed in Examples 12 and 13.

### Example 8—Ultrafiltration/Diafiltration (Hepatitis Antigen pDNA)

This step is another TFF (UF/DF) step using same principle/technique as in Example 3. The loaded product is different: in Example 3, it is clarified supernatant after lysis while here it is the Captobutyl FT (much purer pDNA). The aim is to concentrate the pDNA to about 0.5 mg/ml and to put it in the right buffer (10 mM Tris) to enable the forthcoming BspQ1 enzymatic digestion (linearization).

The 2^{nd} TFF set up was the same as established in run E39. In this step the concentration can be first adjusted to a desired value for further processing, such as 0.3 to 0.8 mg/ml, 0.4 to 0.7 mg/ml or 0.5 to 0.6 mg/ml. Then, ultrafiltration/diafiltration is performed using a technique such as tangential flow filtration.

### Example 9—Collection and/or Storage of Final Product (Hepatitis Antigen pDNA)

The Captobutyl flow through from Example 8 was subjected to further processing according to the procedures reported in Table 3 above. The retentate of the previous TFF is collected and the TFF system is drained

The product is aliquoted into a convenient volume (whenever possible per 40 mL as it is the quantity required for an IVT synthesis) and each container is stored in a −20° C. freezer. The product is ready for next step (linearization by restriction enzyme digestion).

### Example 10—Testing Process with Different pDNA Constructs

As discussed above, Examples 1-9 were performed with pDNA constructs (grown in E. coli) that encode a SAM molecule that encodes a Hepatitis antigen (Except Run (E11-a-e) in Example 5 where SAM-COVID plasmid was used. Therefore, the following experiment was performed on the purification of 50 g of paste (approximately 1 L harvest) of the following four SAM pDNA constructs; Hepatitis antigen; COVID-19; Rabies and HSVth. These plasmids were similar, with a fundamental difference being the immunogen that is encoded. However, these plasmids also had different sizes, charges and possibly conformations (shapes).

FIG. 8 shows that the Hepatitis antigen, COVID, Rabies and HSVth plasmid samples that were subjected to further testing moved differently in an agarose gel. FIG. 8 shows mainly scDNA plasmid form for all batches (intense “smiling” band), few ocDNA visible for hepatitis, covid and hsvth, pattern for rabies is different (larger plasmid and HMW smear visible) and less ocDNA visible on gel for disclosed process material compared to commercial kit

The large gel containing 6 lanes shows that the four pDNA samples (lanes 2-5) were mainly scDNA (the intense “smiling band”). Few ocDNA were visible for Hepatitis antigen, COVID and HSVth (lanes 2, 3 and 5). The pattern for rabies (lane 4) was different, due to the larger plasmid size and a HMW smear is visible above the scDNA band. The smaller gel on the right (containing 2 lanes) compares the COVID sample (E45-COVID-19) with a commercial kit (EndoFree Plasmid Purification Giga Kits built by Qiagen) of scDNA. Another suitable kit could be NucleoBond PC 1000 EF kit built by Macherey Nagel.

Purification was performed on all samples under the conditions generally described in the above runs with Hepatitis antigen pDNA in Examples 1-9, without any adaptation of DSP upfront. The products tested were the products after the 2^{nd} (and last) TFF, namely purified plasmid scDNA. No further purification was performed after this step. All purifications were successful from a processability point of view, i.e., no stop happened. Differences in results observed described below are likely due to the differences in size, charge and conformations of the plasmids.

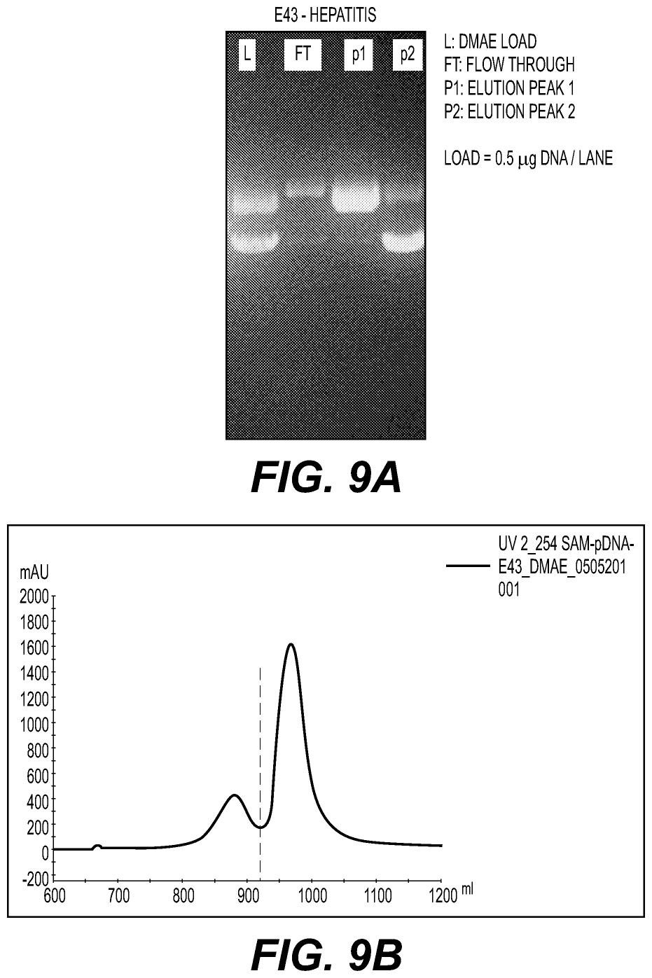

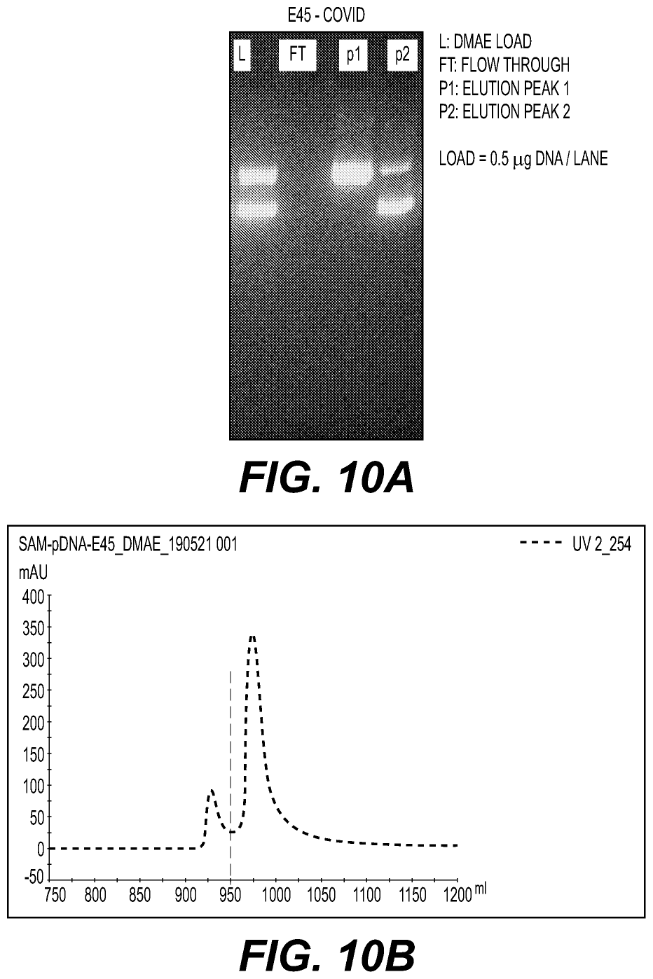

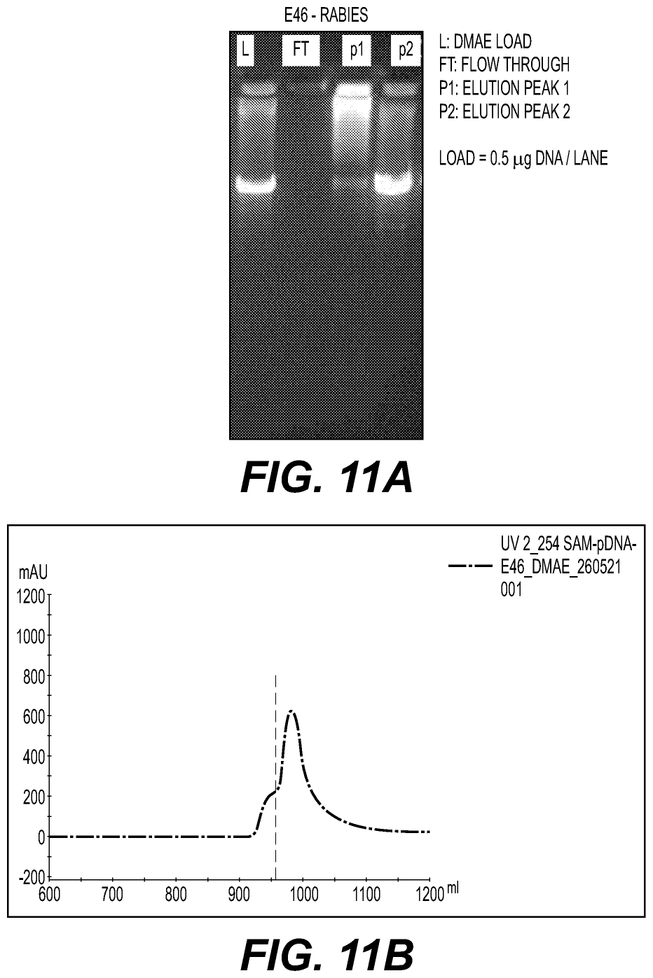

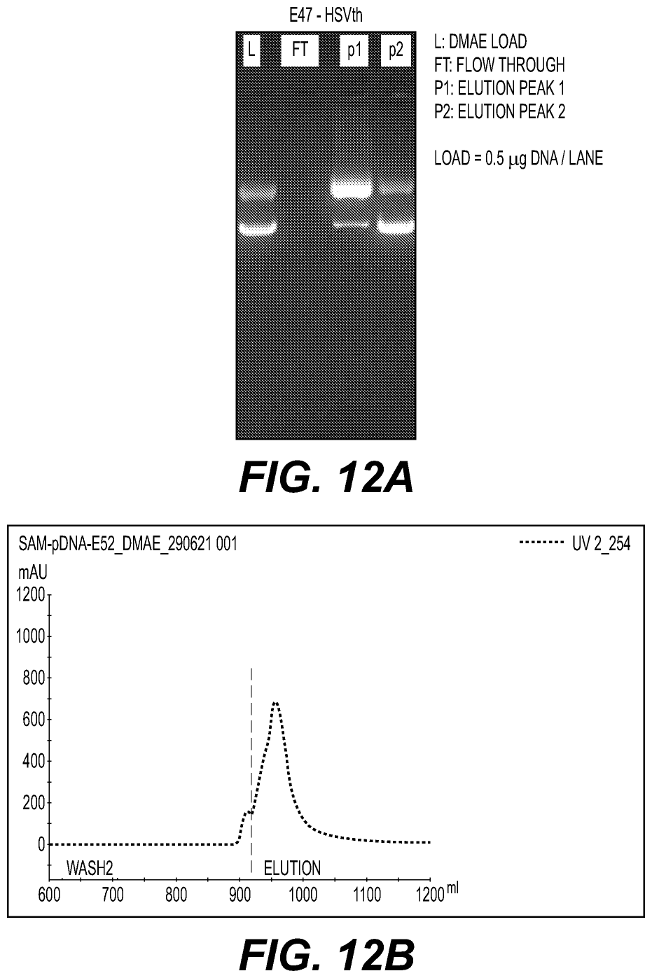

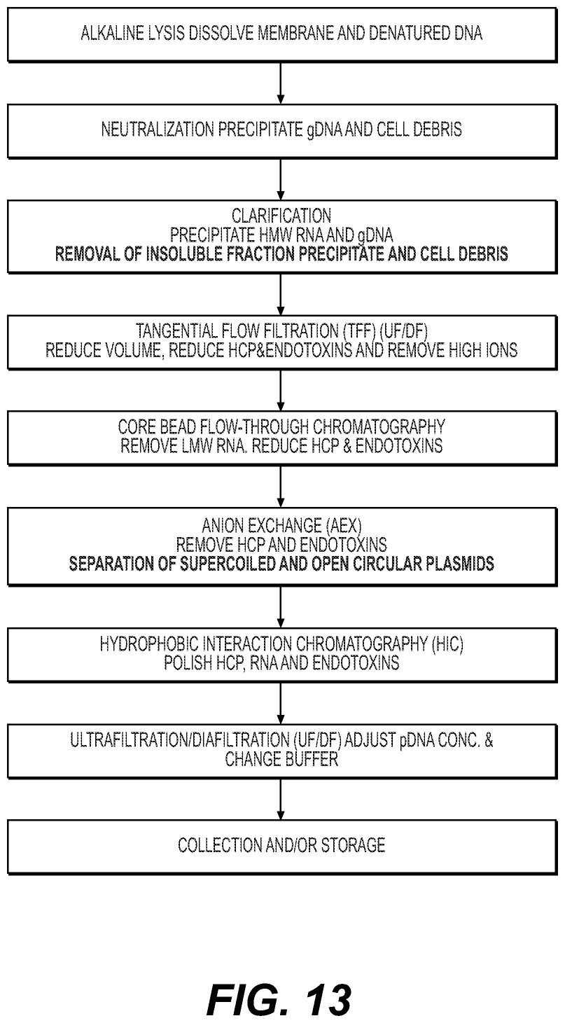

The results obtained for these plasmids as well as the commercial control are shown in FIG. 9A and FIG. 9B (Hepatitis antigen), FIG. 10A and FIG. 10B (COVID); FIG. 11A and FIG. 11B (Rabies); and FIG. 12A and FIG. 12B (HSVth). Similar patterns were observed for HV, COVID and HSV (oc and sc forms are separated in 2 peaks: oc in peak 1 and sc in peak 2). Different patterns were observed for Rabies (no band for ocDNA but a HMW smear abounding in fronting of the peak (peak 1)). It is noted that the fraction of interest (scDNA) is always the later eluted peak.

TABLE 9
Process Performance Benchmark

| | | mg DNA/L | | % | % | Endotox. |
|---|---|---|---|---|---|---|
| | Construct | Harvest | A260/ | Protein | RNA | EU/mg |
| Batch # | (plasmid size, kbp) | (50 g paste) | A280 | residual | residual | DNA |
| Indicative | | >1.5 | 1.80-2.00 | ≤4.0 | ≤7.0 | ≤40 |
| Target (IT) | | | | | | |
| Preferred IT | | >3.0 | 1.80-2.00 | ≤3.0 | ≤6.0 | ≤20 |
| More | | >6.0 | 1.80-2.00 | ≤2.0 | ≤5.0 | ≤10 |
| Preferred IT | | | | | | |
| Ref - scDNA | Hepatitis antigen | 21.6 | 1.89 | 0.9 | 3.7 | 0.5 |
| (commercial | (12 kbp) | | | | | |
| kit machinery) | | | | | | |
| E43 | Hepatitis antigen | 15.0 | 1.91 | 1.6 | 1.6 | 26.0 |
| | (12 kbp) | | | | | |
| E45 | COVID (1^{st} ferm) | 1.9 | 1.90 | 1.7 | 4.9 | 11.5 |
| | (13.8 kbp) | | | | | |
| E46 | Rabies (11.6 kbp) | 6.5 | 1.91 | 4.0 | 3.9 | 7.9 |
| E47 | HSVth (>12 kbp) | 7.0 | 1.91 | 0.5 | 0.7 | 39.6 |
| E52 | COVID (2^{nd} ferm) | 6.2 | 1.90 | 0.8 | 6.1 | 8.4 |
| | (13.8 kbp) | | | | | |

### Productivity:

Hepatitis antigen is 2× above average
HSVth, Rabies and COVID (2^{nd} fermentor) are consistent and in middle of range (6-7 mg pDNA/L harvest). Very first fermentor (COVID 1^{st} ferm) was 3× under average.
6 mg/L is a minimum target as a process performance indicator.

### Purity:

Ratio A260/A280 very similar
Slight discrepancies between constructs for Protein and RNA content (i.e. Protein a bit higher for Rabies and RNA a bit higher for COVID)
Endotox level a bit high compared to target value mostly for HSV. Ideally to further improve process to get 1 log lower

### Remarks:

scDNA is an intermediate product: Target values aligned with performance of commercial kits were used to achieve the development, but these are not specifications as such. Moreover, after linearization, further steps such as another Captocore+UF step might improve purity (not verified).

Analytical values for the 6 batches below were averaged for the 5 assays indicated below:

### Six Batches

E40 (Hepatitis Antigen)
E43 (Hepatitis Antigen)
E45 (COVID)
E46 (Rabies)
E47 (HSVth)
E52 (COVID)

### The 5 Assays

Nanodrop (Abs 254 nm)
DNA content by Qubit assay (total DNA content)
RNA content by Qubit assay (total RNA)
Protein content by pBCA assay
Endotoxin content by Endosafe (LAL assay)

The following values were calculated for the following intermediate process steps:
Total Lysate=after alkaline Lyse+neutralization
SNC=after CaCl₂) and centrifugation (values in supernatant)
UF1-R=retentate sample after the 1^{st} TFF
Capto FT=Flowthrough sample of the Captocore 700
DMAE-P2=pool of peak #2 (scDNA) of DMAE elution gradient
Captobutyl FT=Flowthrough sample of the Captobutyl
pDNA=Retentate after 2^{nd} TFF (final product)

TABLE 10
Average Values (6 batches/4 plasmid types)

| | Nanodrop | Qubit | Qubit | Proteins | Endotoxins |
|---|---|---|---|---|---|
| | (A254) | DNA | RNA | by μBCA | (LAL) |
| Step | μg/ml | μg/ml | μg/ml | μg/ml | EU/ml |
| Total Lysate | 3526 | 216 | 1997 | 28298 | N/A |
| SNC | 415 | 10 | 75 | 7694 | 7276 |
| UF1-R | 357 | 111 | 18 | 2354 | 389 |
| Capto FT | 166 | 98 | 11 | 13 | 126 |
| DMAE-P2 | 92 | 60 | 5.4 | 1.8 | 10.3 |
| Captobutyl FT | 45 | N/A | 1.6 | 1.2 | 1.0 |
| pDNA | 297 | 304 | 6.6 | 5.4 | 7.4 |

### Remarks

between Captobutyl FT and pDNA, there is a concentration of the sample (about 6.5×), which explains why some values can be higher after the UF compared to before.
“N/A” indicates that it is not possible/relevant to perform the assay at that stage (Endotoxin is not measurable in the lysate as there is really too much endotoxin—the assay is not accurate. The Qubit DNA assay is not working in the Captobutyl FT sample due to high NaCl concentration that causes interference).
Values for different variations on the process and/or for different pDNA will differ from the above values. For example, ranges for each of the above values (each value being arbitrarily designated as “X”) can be 0.1 to 10 X, preferably 0.2 to 5 X and more preferably 0.5 to 2 X

### Example 11—Comparison of with Commercial Plasmid Purification Kit by Pulsed-Field Gel Electrophoresis

To compare the efficiency of both the commercialized kit (resulting sample: PEK2201) and the previously described plasmids described in Examples 1-10, plasmid purification process (resulting sample: pDNA-E113, which uses the same PEK2201 plasmid), the following experiment was performed. Samples (purified pDNA) were previously obtained from the results of the last step of the aforementioned process described herein Examples 1-10 to that of a commercialized kit (Qiagen Gigaprep).

The results from the comparison of purity of pDNA product are shown in Table 11 below. Similar purity can be seen between both strategies as denoted by their ratio A260/A280, Residual Protein and Endotoxin content results.

TABLE 11

| | | | [DNA] | [RNA] | | QDNA | Residual | |
|---|---|---|---|---|---|---|---|---|
| | | Ratio | μg/ml | μg/Ml | relative | mg/L | Protein | Endotoxin |
| Sample | Process | A260/A280 | Nanodro | Qubit | % RNA | Harv | % | EU/mgDNA |
| PEK2201 | QIAGEN | 1.90 | 567 | 59.8 | 9.9 | 28.3 | 0.7 | 11.4 |
| RSV | Gigaprep | | | | | | | |
| pDNA-E113 | Present | 1.91 | 718 | 29.5 | 3.5 | 23.8* | 0.5 | 9.7 |
| RSV | Process | | | | | | | |

Pulsed-field gel electrophoresis (PFGE) was then performed on both samples to determine the proportion of open circular conformations as compared to the supercoiled conformations which separates based on the different electrophoretic ability.

PFGE is an improved electrophoretic method enabling the separation of high molecular weight DNA molecules with a better size resolution than conventional agarose or acrylamide gel electrophoresis (Maule J, Mol Biotechnol., (1998) “Pulsed-field gel electrophoresis” and Lopez-Canovas, et al, Analytical Biochemistry, (2019) “Pulsed Field Gel Electrophoresis: Past, present, and future.”

Of note, the current process of the claimed invention achieved a higher % of supercoiled plasmid conformations and a corresponding lower % of open circular and no other conformations. This suggests that the purification process discussed in Examples 1-10 is just as efficacious as the commercial kit.

TABLE 12

| | FEMTO-Pulse | | | | FEMTO-Pulse | | | | |
|---|---|---|---|---|---|---|---|---|---|
| | (if SC = multimeric) | | | | (if SC ≠ multimeric) | | | | |
| | | | | QscDNA | | | | | QscDNA |
| | | | % other | (mg/L | | | % other | % | (mg/L |
| Sample | % SC | % OC | conformations | Harv) | % SC | % OC | conformations | multimeric | Harv) |
| PEK2201 | 75.3 | 20.9 | 3.1 | 21.3 | 66.0 | 20.9 | 3.8 | 9.3 | 18.7 |
| pDNA-E113 | 88.0 | 12.0 | 0.0 | 20.9 | 77.2 | 12.0 | 0.0 | 10.9 | 18.4 |

Overall, it can be seen from Tables 11 and 12 that the small-scale process (Examples 1-10) is comparable to the commercially available kit. In both the cases, the purification process yields a high purity and highly homogeneous sc pDNA product.

### Example 12—Optimized Plasmid DNA Purification Process

The optimized larger scale pDNA purification process was performed in accordance with Examples 1-10 but with the following exceptions listed below.

The processes described in Examples 1-10 include the purification of pDNA that encoded a SAM molecule having about 12-18 kbp whereas in this example, classical pDNA which is about 3-fold smaller, was employed, allowing for higher volumetric yield and broader applicability.

In this example, downstream purification activities were utilized to achieve the large-scale purification of pDNA. More specifically, downstream processing methods coupled with anion-exchange chromatography aided in the separation of any unwanted plasmid variants that is ineffective in transferring genetic material and endotoxins (LPS) from the supercoiled plasmid that make up the desired purified pDNA product.

Tables 13-15 summarizes the processing conditions of Day 1-3 of the Optimized Plasmid DNA Purification Process.

TABLE 13
(Day 1)

| | |
|---|---|
| Cell Lysis and | Cells Alkaline Lysis |
| Neutralization | Resuspend 750 g of E. coli paste (equivalent of 15 L harvest collected) in |
| | 7500 mL of 150 mM Tris - 10 mM EDTA - 50 mM Glucose - pH 8.0 |
| | Add 7500 mL of 0.2M NaOH - 1% SDS - Check pH (12.0 ± 0.5) - |
| | Agitate very smoothly during 5 min in STIRRED TANK |
| | Neutralize by adding progressively ~1950 mL of cold 3M potassium |
| | acetate (pH 5.5) to reach a pH between |
| | 7.0-8.0 |
| | Adjust pH to 7.8 ± 0.1 with 1.8M formic acid or NaOH 0.5M |
| Clarification | Precipitation: Add 4M CaCl₂ to reach a final concentration of 0.3M |
| | CaCl₂ - incubate for 15 min |
| | Centrifugation: 20 min at 10 000 × g - 4° C. - collect supernatant |
| | Filtration of the lysate SN through 0.45/0.2 μm Sartobran P filter |
| TFF | Pellicon 3 - 2.28 m² - Biomax 50 kDa |
| (UF/DF) | Concentration: Concentrate filtered SN about 8x to reduce volume |
| | (18 L → 2.2 L) |
| | Diafiltration 1: diafilter 20 DV against 10 mM Tris - pH 7.5 |
| | Diafiltration 2: diafilter 10 DV against 10 mM Tris - 10 mM |
| | EDTA - pH 7.5 |
| | Collect retentate and rinse the system with 10 mM Tris - 10 mM |
| | EDTA - pH 7.5 |
| | Pool retentate and rinse fraction: final Fcc = 8x |

TABLE 14
(Day 2)

| | |
|---|---|
| Multimodal | Captocore 700 (FT MODE) - CV = 800 mL |
| Xto | Sample conditioning: UF retentate is spiked to 0.5M NaCl |
| | with 5M NaCl stock solution |
| | Equilibration: 6 CV of 10 mM Tris - 10 mM EDTA - 0.5M |
| | NaCl - pH 7.5 |
| | Load: Total spiked UF-R (~3 CV) - collect FT |
| | Wash: 5 CV of 10 mM Tris - 10 mM EDTA - 0.5M NaCl - |
| | pH 7.5 - collect 1^{st} CV with FT |
| | Regeneration: 6 CV of 1M NaOH - 30% isopropanol including a static |
| | pause of 1 h after 3 CV |
| AEX | Fractogel EMD DMAE (BIND-ELUTE MODE) - CV = 450 mL |
| Xto | Equilibration: 4 CV of 25 mM Tris - 1 mM EDTA - 0.5M NaCl - pH 7.5 |
| | Load: Total of the Captocore FT |
| | Wash 1: 4 CV of 25 mM Tris - 1 mM EDTA - 0.5M NaCl - pH 7.5 |
| | Wash 2: 3 CV of 25 mM Tris - 1 mM EDTA - 0.65M NaCl - pH 7.5 |
| | Elution Gradient: from A: 25 mM Tris - 1 mM EDTA - 0.65M NaCl pH |
| | 7.5 to B: 25 mM Tris - 1 mM EDTA - 1.0M NaCl - pH 7.5 on 15 CV: |
| | collect 2^{nd} peak or 2^{nd} part of the peak |
| | Regeneration: 5 CV of 0.5M NaOH - 2M NaCl |

TABLE 15
(Day 3)

| | |
|---|---|
| HIC | Captobutyl (FT MODE) - CV = 450 mL |
| Xto | Sample conditioning: DMAE eluate is spiked to 1.7M (NH4)₂SO4 |
| | Equilibration: 5 CV of 10 mM Tris - 1 mM EDTA - 1.7M (NH4)₂SO4- pH |
| | 7.5 |
| | Load: Total of spiked DMAE eluate- collect FT |
| | Wash 1: 5 CV of 10 mM Tris - 1 mM EDTA - 1.7M (NH4)₂SO4- pH 7.5 - |
| | collect 1^{st} CV with FT |
| | Wash 2: 6 CV of 10 mM Tris - 1 mM EDTA - pH 7.5 |
| | Regeneration: 5 CV of 10 mM Tris - 50% Ethylene glycol |
| TFF | Pellicon 3 - 0.11 m² - Biomax 30 kDa |
| (UF/DF) | Concentration: Concentrate the Catpobutyl FT to reach a concentration of 0.5 |
| | mg/ml (monitoring with Nanodrop) |
| | Diafiltration: diafilter 10 DV against 10 mM Tris - pH 8.0 |
| | Collect retentate → purified plasmid fraction (pDNA) |
| Storage | Storage of pDNA at −20° C. |

750 g of E. coli cells were lysed and neutralized using the conditions reported in Table 13 to prepare a neutralized cell lysate. It is known in the art that insufficient mixing can generally result in local pH extremes, causing irreversibly denatured plasmid (U.S. Pat. No. 9,725,725 B2). To perform batch mixing, the standard manual mixing was not possible at a large-scale because of the large volumes to be mixed and the lack of reproducibility from person to person. A specialized stirred tank consisting of three baffles and two low power number impellers (for gentle mixing) was used to mechanically mix the large lysis volume. Implementation of the stirred tank allowed for a more homogenous solution, improving lysis of the product and its subsequent pDNA yield, which is more convenient for bulk purification of high quality pDNA.

pDNA homogeneity was visualized and compared using Lyse blue to determine level of homogeneity and the resultant productivity of the harvested lyse conditions are shown in Table 16. As reiterated above, insufficient mixing can result in local pH extremes, causing irreversibly denatured plasmid and also result in incomplete lysis due to NaOH-SDS solution not being able to reach all of the cells which subsequently results to loss of yield. Further, aggressive mixing can damage pDNA and fragment gDNA (U.S. Pat. No. 9,725,725 B2). As seen in Table 16, the stirred tank allowed for better mixing of product which resulted in almost 3-fold better yield as compared to manual lysis which is integral to the efficacy of the large-scale production process.

Table 16 showing Comparison of Lyse Conditions

| | Visual | End of Purification |
|---|---|---|
| Lyse Condition | Result | Harvest |
| Manual Lysis | Not homogenous | 4.4 mg/L |
| (Duran Schott 4x | | |
| upside-down) | | |
| Orbital Shaker | Partially homogenous | ~12.4 mg/L |
| (PETG 2 L) | Not convenient for SU | |
| Stirred Tank 1.5 L | Most homogenous | 11.3 mg/L |
| (200 rpm) | **Convenient for SU | |

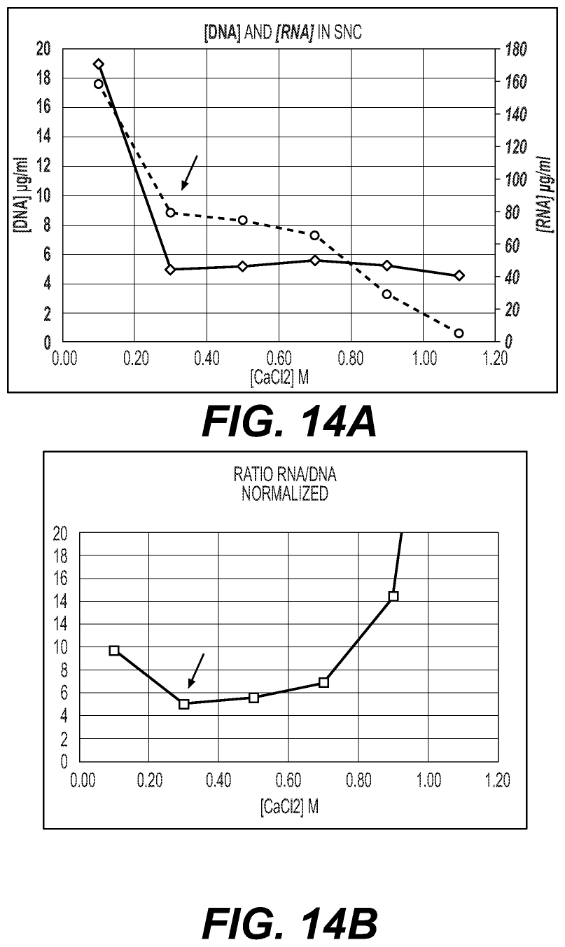

The neutralized cell lysate obtained was clarified by precipitation, centrifugation and filtration using the optimized conditions reported in Table 13 to prepare a clarified composition. In this clarification step, 4M CaCl₂) was added directly to the neutralized cell lysate to bring the CaCl₂) concentration to 0.3 M and a single centrifugation step was employed at a lower centrifugation speed (10,000 g) and shorter time (20 minutes). As shown in FIG. 14, the optimal concentration of 0.3M precipitated the most RNA which is indicative of the lowest ratio of RNA/DNA.

TFF was performed on the clarified composition under the conditions of Table 13 to produce a retentate containing 2200 ml containing the retained pDNA and all other undesired impurities which are then separated via Core Bead Flow-Through Chromatography. The resulting flowthrough containing the desired pDNA was then subjected to AEX as described in Example 4 but with the optimal conditions described in Table 14.

Multimodal chromatography is more advantageous over traditional chromatography due to its enhanced selectivity for isolating pDNA in the pDNA purification process. In this step, sc pDNA is isolated using a gradient NaCl elution method that includes elution of sc pDNA, washing of weakly bound contaminants and removal of other RNA impurities.

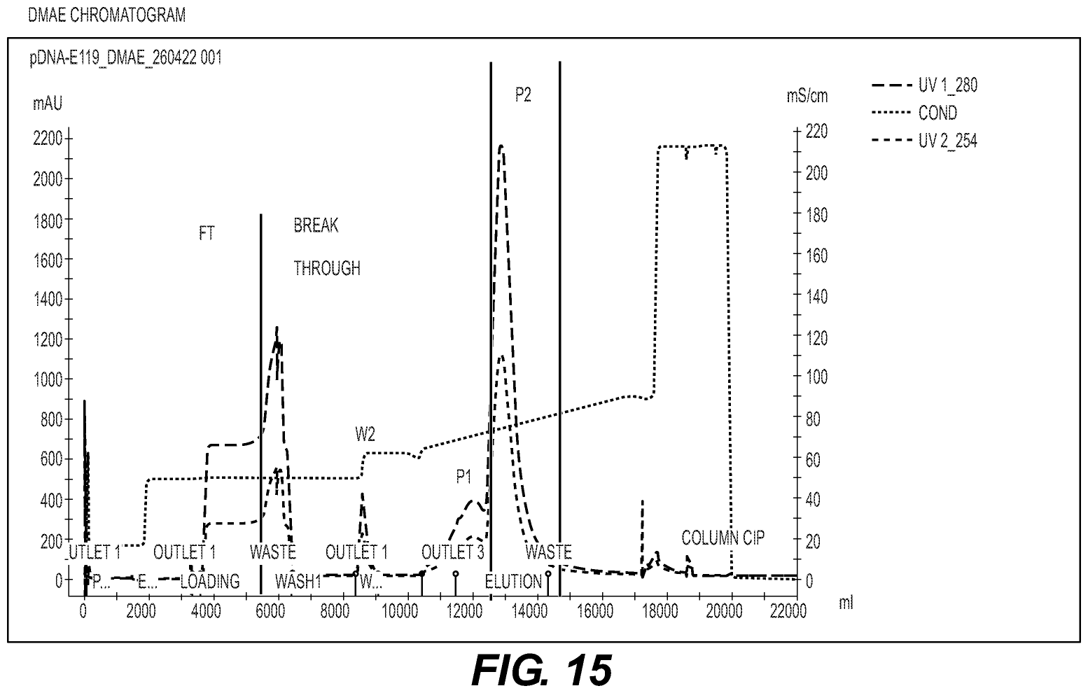

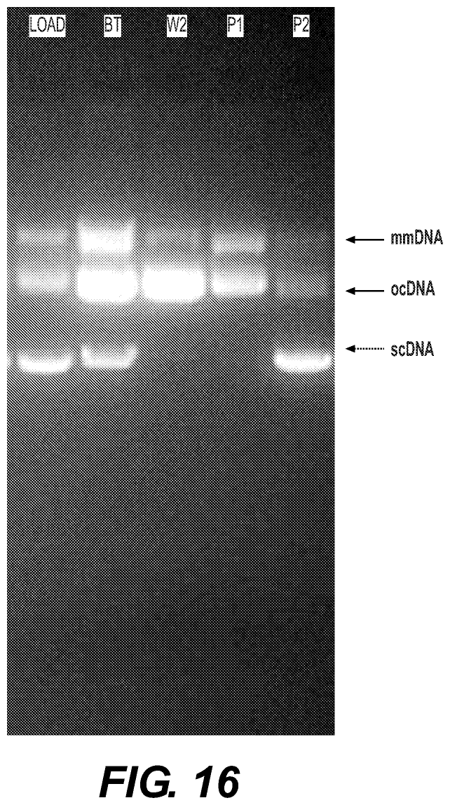

AEX is then characterized by two distinct peaks where the OC plasmid isoform which has lower charge density elutes earlier than the more compact SC pDNA due to their higher charge density. The separated fractions were analyzed and the elution gradient results for the Fractogel EMD DMAE on the chromatogram in FIG. 15. Based on this data, each peak was individually collected where peak 1 eluted oc DNA and peak 2 eluted scDNA and later analyzed and confirmed on the agarose gel shown as FIG. 16.

The purified scDNA collected from peak 2 of the AEX step was further treated with Captobutyl to remove any remaining impurities in this HIC step. Captocore FT samples were used and spiked with 1.7 M ammonium sulphate alone. In this case, ammonium sulphate was chosen to be the better salt for achieving the large batch purification because it effectively removed more endotoxins as compared to using NaCl alone or in combination thereof.

Table 17 Showing Effects of Varying Salts and Concentrations Required for HIC Step on the Resultant pDNA Yield

| | | CaptoButyl | Endotoxin | pDNA Yield |
|---|---|---|---|---|
| | Plasmid | Salt type and | content in | (%) for |
| Run # | strain | concentration | HIC-FT (EU/ml) | HIC step |
| pDNA-E44 | SAM-HBVth | 3M NaCl | 15.50 | 93.6% |
| pDNA-E61A | SAM-HBVth | 1.5M (NH4)2SO4 | 1.14 | 103.5% |
| pDNA-E61B | SAM-HBVth | *1.7M (NH4)2SO4 | 0.64 | 103.3% |
| pDNA-E101 | pDNA-COVID | 1.85M (NH4)2SO4 | 1.67 | 97.1% |

To achieve the large-scale purification of pDNA, ammonium sulphate was used in in this HIC step because it provided a better endotoxin clearance (at least 10×lower than that of NaCl) and excellent % pDNA yield (no loss of product observed on the HIC column whilst 5-10% loss was observed with NaCl) as shown in the above Table 17. The optimal concentration of 1.7M (NH4)2SO4 was therefore chosen to replace NaCl in the HIC experiment (as opposed to the experiment done in Example 8).

Captobutyl FT was then subjected to its final TFF (UF/DF) step according to the conditions of Table 15. To concentrate such large quantities of much purer pDNA, the diafiltrate was reduced to 10 DV 10 mM Tris and the resultant retentate collected and stored. Approximately, 200-400 mg of sc pDNA was obtained from 750 g of E. Coli as compared to the 20 mg usually obtained in the small-scale process.

Comparison between small-scale (Examples 1-10) and large-scale (this Example 12) batch production were also compared in Table 18 to show that both processes resulted in similar productivity and purity between them, hereby confirming that the optimized procedure was efficient at purifying bulk purified pDNA of great quality in a shorter time frame.

### Table 18 Showing Small-Scale Vs Large-Scale Purity and Productivity

| | pDNA-E118 (pDNA081) | pDNA-E119 (pDNA081) |
|---|---|---|
| | (OMICRON - 50 g paste) | (OMICRON - 750 g paste) |
| Productivity | 27 mg pDNA/L | 27.3 mg pDNA/L |
| | Harvest (1) | Harvest (1) |
| Ratio A260/A280 | 1.94 | 1.91 |
| RNA content by Qubit | 0.6% | 1.5% |
| Protein content by μBCA | 0.5% | 0.6% |
| Endotoxin content by Endosafe | 0.8 EU/mg | 1.8 EU/mg |

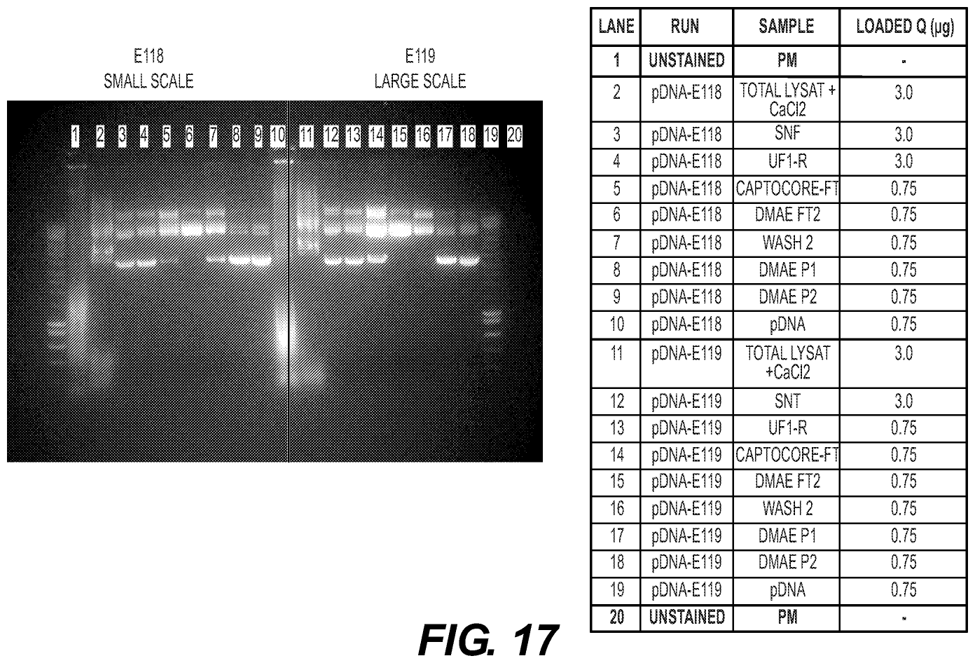

The data shown in Table 18 emphasizes that the optimized purification process as adapted with a more functional mixing strategy, and optimized concentrations of excipients allows for a reproducible and most importantly, scalable process that produces similar high quality pDNA even at such a large-scale. Further, FIG. 17 shows the quantity of pDNA eluted at integral steps of both the small-scale and large-scale process. They both show similar distribution of pDNA highlighting that the optimized process while scalable, does not compromise on pDNA integrity and quality.

Collectively, these results support the small-scale process can serve as a predictive tool for what is occurring at a large-scale.

NOTE: The Plasmid Sizes for the Conventional mRNA Tested are:
pXW02-C23—4165 bp; “empty” plasmid size is 2265 bp, the portion encoding the RNA is 1900 bp
KM70—6250 bp; empty plasmid size is 2265 bp, the portion encoding the Omicron mRNA is 3985

### Example 13—Further Comparison of Optimized Plasmid DNA Purification Processes (SS and LS) with Commercial Plasmid Purification Kit by Pulsed-Field Gel Electrophoresis

The small-scale pDNA purification process of Examples 1-10 was optimized according to the same conditions of the scalable process of Example 12 as shown below in Table 19.

Table 19 highlights the changes therein to the small-scale process including changes to reagents where applicable, concentrations of excipients where applicable and experimental conditions, respectively. For clarity, more important optimisations are underlined and bolded but comparison for both small-scale processes can always be made directly from Tables 14-16 to Tables 19-21 disclosed below. For example, centrifugation time was shortened in the clarification step and ammonium sulphate was used instead of NaCl in the HIC step for improved endotoxin clearance.

TABLE 19
(Day 1)

| | |
|---|---|
| Cell Lysis and | Cells Alkaline Lysis |
| Neutralization | Resuspend 50 g of E. coli paste (equivalent of 1 L harvest collected) in |
| | 500 mL of 150 mM Tris - 10 mM EDTA - 50 mM Glucose - pH 8.0 |
| | Add 500 mL of 0.2M NaOH - 1% SDS - Check pH (12.0 ± 0.5) - |
| | Agitate very smoothly during 5 min in STIRRED TANK |
| | Neutralize by adding progressively ~130 mL of cold 3M potassium |
| | acetate (pH 5.5) to reach a pH between |
| | 7.0-8.0 |
| | Adjust pH to 7.8 ± 0.1 with 1.8M formic acid or NaOH 0.5M |
| Clarification | Precipitation: Add 4M CaCl₂ to reach a final concentration of 0.3M |
| | CaCl₂ - incubate for 15 min |
| | Centrifugation: 20 min at 10 000 × g - 4° C. - collect supernatant |
| | Filtration of the lysate SN through 0.45/0.2 μm Sartobran P filter |
| TFF | Pellicon 3 - 0.11 m² - Biomax 50 kDa |
| (UF/DF) | Concentration: Concentrate filtered SN about 8x to reduce volume |
| | (1200 mL → 150 mL) |
| | Diafiltration 1: diafilter 20 DV against 10 mM Tris - pH 7.5 |
| | Diafiltration 2: diafilter 10 DV against 10 mM Tris - 10 mM |
| | EDTA - pH 7.5 |
| | Collect retentate and rinse the system with 10 mM Tris - 10 mM |
| | EDTA - pH 7.5 |
| | Pool retentate and rinse fraction: final Fcc = 8x |

TABLE 20
(Day 2)

| | |
|---|---|
| Multimodal | Captocore 700 (FT MODE) - CV = 53 mL |
| Xto | Sample conditioning: UF retentate is spiked to 0.5M NaCl with 5M |
| | NaCl stock solution |
| | Equilibration: 6 CV of 10 mM Tris - 10 mM EDTA - 0.5M NaCl - pH |
| | 7.5 |
| | Load: Total spiked UF-R (~3 CV) - collect FT |
| | Wash: 5 CV of 10 mM Tris - 10 mM EDTA - 0.5M NaCl - pH 7.5 - |
| | collect 1^{st} CV with FT |
| | Regeneration: 6 CV of 1M NaOH - 30% isopropanol including a static |
| | pause of 1 h after 3 CV |
| AEX | Fractogel EMD DMAE (BIND-ELUTE MODE) - CV = 30 mL |
| Xto | Equilibration: 4 CV of 25 mM Tris - 1 mM EDTA - 0.5M NaCl - pH 7.5 |
| | Load: Total of the Captocore FT |
| | Wash 1: 4 CV of 25 mM Tris - 1 mM EDTA - 0.5M NaCl - pH 7.5 |
| | Wash 2: 3 CV of 25 mM Tris - 1 mM EDTA - 0.65M NaCl - pH 7.5 |
| | Elution Gradient: from A: 25 mM Tris - 1 mM EDTA - 0.65M NaCl pH |
| | 7.5 to B: 25 mM Tris - 1 mM EDTA - 1.0M NaCl - pH 7.5 on 15 CV: |
| | collect 2^{nd} peak or 2^{nd} part of the peak |
| | Regeneration: 5 CV of 0.5M NaOH - 2M NaCl |

TABLE 21
(Day 3)

| | |
|---|---|
| HIC | Captobutyl (FT MODE) - CV = 30 mL |
| Xto | Sample conditioning: DMAE eluate is spiked to 1.7M (NH4)₂SO4 |
| | Equilibration: 5 CV of 10 mM Tris - 1 mM EDTA - 1.7M (NH4)₂SO4- |
| | pH 7.5 |
| | Load: Total of spiked DMAE eluate- collect FT |
| | Wash 1: 5 CV of 10 mM Tris - 1 mM EDTA - 1.7M (NH4)₂SO4- pH 7.5 - |
| | collect 1^{st} CV with FT |
| | Wash 2: 6 CV of 10 mM Tris - 1 mM EDTA - pH 7.5 |
| | Regeneration: 5 CV of 10 mM Tris - 50% Ethylene glycol |
| TFF | Pellicon 3 - 88 cm² - Biomax 30 kDa |
| (UF/DF) | Concentration: Concentrate the Catpobutyl FT to reach a |
| | concentration of 0.5 mg/ml (monitoring with Nanodrop) |
| | Diafiltration: diafilter 10 DV against 10 mM Tris - pH 8.0 |
| | Collect retentate → purified plasmid fraction (pDNA) |
| Storage | Storage of pDNA at −20° C. |

All of the samples in the below Table 22 (except QIAGEN and pDNA-E119) were produced with the process described in Tables 14, 15 and 16 below, except that the quantity surfaces and volumes are smaller for the small-scale batches. pDNA-E119 was produced by the process described in in Table 14, including Quantity Surface and Volumes. The batch PEK2201 (used as a benchmark) was produced using a commercial QIAGEN extraction kit, using the same starting paste as pDNA-E113.

Based on the aforementioned details, comparison analyses were then conducted to compare the quality of pDNA product between the three processes similar to the PFGE experiment of Example 11. Of interest, the analyses revealed similar trends to that of Table 12 as evidenced by the results of Table 22.

TABLE 22

| | | | | | | | FEMTO Pulse data (ARD result - | | | | |
|---|---|---|---|---|---|---|---|---|---|---|---|
| | | | | | | | O. Vandeputte/G. Bayon-Vicente) | | | | |
| | | | | | | | | | % SC | | |
| | | | Sample | | | | % SC | % SC | (Mono + | | % |
| Process | Step | #purif | Name | Antigen | Plasmid | [pDNA] | (monomeric) | Multimeric | Multi) | % OC | Other |
| SS | ScDNA | pDNA- | pDNA- | RSV | pXW02- | 0.45 | 78.1 | 10.0 | 88.1 | 11.9 | 1.0 |
| | | E112 | E112 | | C23 | mg/ml | | | | | |
| | | | UF2-R | | | | | | | | |
| SS | scDNA | pDNA- | pDNA- | RSV | pXW02- | 0.72 | 77.2 | 10.9 | 88 | 12 | 0.0 |
| | | E113 | E113 | | C23 | mg/ml | | | | | |
| | | | UF2-R | | | | | | | | |
| QIAGEN | ScDNA | PEK2201 | PEK2201 | RSV | pXW02- | 0.57 | 66 | 9.3 | 75.3 | 20.9 | 3.1 |
| | | | 5x diluted | | C23 | mg/ml | | | | | |
| SS | scDNA | pDNA- | pDNA- | OMICRON | KM70 | 0.73 | 77.6 | 15.4 | 93 | 6.6 | 0.6 |
| | | E118 | E118 | | | mg/ml | | | | | |
| | | | UF2-R | | | | | | | | |
| LS | scDNA | pDNA- | pDNA- | OMICRON | KM70 | 0.50 | 77.9 | 12.4 | 90.3 | 8.5 | 1.2 |
| | | E119 | E119 | | | mg/ml | | | | | |
| | | | pDNA | | | | | | | | |
| LS | scDNA | pDNA- | pDNA- | OMICRON | KM70 | 0.47 | 60.4 | 28.1 | 88.5 | 11.3 | 0.6 |
| | | E120 | E120 | | | mg/ml | | | | | |
| | | | pDNA | | | | | | | | |

In the above Table, SS means a small-scale process was used and LS means a larger-scale process was used.

As shown in Table 22, the overall range of % OC remains between 7% and 12% for all the batches produced with the process of Table 19-21 (small-scale; batch pDNA E112, pDNA E113 and pDNA E118) whereas the commercial kit obtained about 21% OC accordingly. Comparatively, since the large scale has proven to be indicative of the small-scale process, the data demonstrates that the overall range of % OC obtained from the scalable process of Table 14-16 (large-scale; batch pDNA E119 and pDNA E120) was between 8-12% which is comparative and lower than that of the commercial kit as well. Subsequently, the % of SC for the small-scale process is also higher ranging between 88-93% and 88-90% for all large-scale samples, respectively, whereas that of the commercial kit is 75%.

Overall, this data highlights great promise for the scalable process described herein.

1. A method of purifying plasmid DNA (pDNA), comprising the steps of:
subjecting a sample comprising pDNA to a core bead flow-through chromatography step to reduce the level of at least endotoxin to produce a core bead flow-through; and
subjecting the core bead flow-through to an anion exchange chromatography step.

2. The method of claim 1, wherein the core bead flow-through chromatography removes materials by both size exclusion and binding properties.

3. The method of claim 1, wherein the core bead flow-through chromatography is performed with beads that have an inactive shell containing pores and a core underneath the inactive shell, wherein core ligands located in the core are in fluid communication with the exterior of the beads through said pores.

4. The method of claim 3, wherein said core ligands are both hydrophobic and positively charged.

5. The method of claim 2, wherein the core bead flow-through chromatography is performed using beads comprising a shell containing pores and a core underneath the shell, wherein the pores in the shell have a cut-off at a molecular mass (Mr) of 400 kd or greater and exclude materials having a Mr greater than the cut-off.

6. The method of claim 2, wherein the core bead flow-through chromatography is performed using beads comprising a shell containing pores and a core underneath the shell, wherein the pores in the shell have a cut-off at a molecular mass (Mr) of 600 kd or greater and therefore exclude materials having a Mr greater than the cut-off.

7. The method of claim 2, wherein the core bead flow-through chromatography is performed using beads comprising a shell containing pores and a core underneath the shell, wherein the pores in the shell have a cut-off at a molecular mass (Mr) of 700 kd or greater and therefore exclude materials having a Mr greater than the cut-off.

8. The method of claim 1, wherein a buffer containing sodium chloride is used in the core bead flow-through chromatography step.

9. The method of claim 1, wherein a buffer containing sodium chloride is used in the anion exchange chromatography step.

10. The method of claim 1, wherein a buffer containing sodium chloride is used in the core bead flow-through chromatography step and the concentration of the sodium chloride in the anion exchange chromatography step.

11. The method of claim 10, wherein the concentration of sodium chloride in the buffer used in the core bead flow-through chromatography step and the anion exchange chromatography step are both in the range of 150 mM to 900 mM.

12. The method of claim 1, wherein the sample comprising pDNA is obtained from E. coli cells that have been lysed by alkaline lysis.

13. The method of claim 1, wherein said pDNA encodes a mRNA.

14. The method of claim 1, wherein said pDNA encodes a mRNA of greater than 5,000 bases.

15. The method of claim 1, wherein said pDNA encodes a SAM molecule.

16. The method of claim 1, which is a large-scale batch purification method.

17. A method of improving the quality of a template pDNA prior to an in vitro transcription reaction, comprising the steps of: (i) subjecting a sample comprising pDNA to a core bead flow-through chromatography step to reduce the level of at least endotoxin to produce a core bead flow-through; (ii) subjecting the core bead flow-through to an anion exchange chromatography step; and (iii) collecting the fraction comprising super coiled (sc) pDNA

18. A method for purifying pDNA comprising the steps of: i) lysing a large sample of host cells in a large buffer volume to obtain a cell lysate and treating with a salt to precipitate the RNA to produce a neutralized cell lysate; ii) producing a clarified cell lysate by clarifying the neutralized cell lysate in the appropriate excipient buffer solution and filtering the clarified cell lysate through tangential flow filtration to produce a filtered pDNA sample; iii) subjecting the filtered pDNA sample to a core bead flow-through chromatography step to produce a core bead flow-through; iv) subjecting the core bead flow-through to an anion exchange chromatography step wherein different plasmid DNA isoforms are separated into fractions, and a desired scDNA fraction or fractions are eluted; v) further removing endotoxin impurities from desired scDNA fraction or fractions by subjecting said fraction or fractions to a chromatography step utilizing an HIC resin on a Captobutyl column to produce a Captobutyl eluate; and vi) subjecting said Captobutyl eluate to a second tangential flow filtration step to filter and concentrate the Captobutyl eluate to produce purified pDNA in a form suitable for storage.

19. The method of claim 18, wherein said pDNA purification method comprises at least 2, preferably 3, chromatography steps to achieve a large-scale batch of high-purity pDNA product.

20. The method of claim 18, wherein in said step (i) the lysing step comprises use of an alkali salt and an ionic detergent.

21. The method of claim 18, wherein in said step (i) the lysing step comprises agitating the cell lysate in a stirred tank to achieve high-purity pDNA homogeneity.

22. The method of claim 18, wherein in said step (v) the scDNA product is spiked with ammonium sulphate.

23. The method of claim 18, wherein in said step (i) the volume of said culture is at least 15 liters.

24. The method of claim 18, wherein in said step (ii), the excipient is CaCl₂.

25. A plasmid DNA composition comprising pDNA wherein at least 80% of the plasmid DNA is in supercoiled form, less than 15% is in open-circular form and less than 5% is in other isoforms, all percentages being based on the total amount of pDNA present.

26. The plasmid DNA composition according to claim 25 comprising no more than 15% nicked pDNA during separation of different plasmid isoforms present in the clarified lysate.

27. A method for synthesizing RNA comprising using the pDNA produced according to the method of claim 1, in an in vitro transcription reaction to synthesize the RNA.

28. The pDNA produced according to the method of claim 1, wherein said pDNA is suitable for pharmaceutical use.

29. A method for synthesizing RNA comprising using the pDNA produced according to the method of claim 18 or the plasmid of claim 25, in an in vitro transcription reaction to synthesize RNA.

30. The pDNA produced according to the method of claim 18, wherein said pDNA is suitable for pharmaceutical use.

31. A method for synthesizing RNA comprising using the plasmid of claim 25, in an in vitro transcription reaction to synthesize the RNA.
